(19) United States
(12) Patent Application Publication
(10) Pub. No.: US 2024/0249802 A1
(43) Pub. Date: Jul. 25, 2024

(54) TRAINING METHOD AND APPLICATION METHOD FOR REACTION PRODUCT PREDICTION MODEL, APPARATUS, AND DEVICE

(71) Applicant: TENCENT TECHNOLOGY (SHENZHEN) COMPANY LIMITED, Shenzhen (CN)

(72) Inventors: Ziqiao MENG, Shenzhen (CN); Peilin ZHAO, Shenzhen (CN)

(21) Appl. No.: 18/624,349

(22) Filed: Apr. 2, 2024

Related U.S. Application Data
(63) Continuation of application No. PCT/CN2023/089401, filed on Apr. 20, 2023.

(30) Foreign Application Priority Data
Jul. 14, 2022 (CN) 202210826462.3

(51) Int. Cl. G16C 20/70; G16C 20/10

(52) U.S. Cl. CPC G16C 20/70; G16C 20/10

(57) ABSTRACT

Embodiments of this application disclose a training method and an application method for a reaction product prediction model. The method includes constructing a positive sample reactant set and a negative sample reactant set through a first auxiliary network; determining a reaction predicted loss value; constructing a positive sample reaction group set and a negative sample reaction group set through a second auxiliary network; determining a reaction relationship predicted loss value; determining, through a third auxiliary network according to the sample reactant vector and the sample reaction product vector, an atom label and a predicted probability value of an atom in the sample reactant being present in a main product; determining an atom predicted loss value; and training the reaction product prediction model based on the reaction predicted loss value, the reaction relationship predicted loss value, and the atom predicted loss value, to obtain a target reaction product prediction model.

### RELATED APPLICATIONS

This application is a continuation of PCT Application No. PCT/CN2023/089401, filed on Apr. 20, 2023, which claims priority to Chinese Patent Application No. 2022108264623, filed with the China National Intellectual Property Administration on Jul. 14, 2022, and entitled “TRAINING METHOD AND APPLICATION METHOD FOR REACTION PRODUCT PREDICTION MODEL, APPARATUS, AND DEVICE”. The two applications are both incorporated herein by reference in their entirety.

### FIELD OF THE TECHNOLOGY

This application relates to the field of artificial intelligence technologies, and in particular, to a reaction product prediction technology.

### BACKGROUND OF THE DISCLOSURE

Predicting organic chemical reaction products is significant to technical fields such as computational chemistry and pharmaceuticals.

A conventional organic chemical reaction prediction method relies on common reaction templates to predict possible product structures. However, there are many types of organic chemical reactions, and new reactions are emerging as chemical research become increasingly sophisticated. Consequently, it is difficult for the reaction templates to cover all reaction types, and the reaction templates cannot be applied to new reaction types. With the development of deep learning technology, it is particularly important to use the deep learning technology to learn potential reaction rules from organic chemical reaction data.

However, currently used technology of performing deep learning based on organic chemical reaction data generally relies on a large quantity of manual labels to train reaction product prediction models. Moreover, to manually label data is usually very expensive, and as the data amount of chemical reaction data increases, support from more manual labels is usually needed, which consumes a lot of labor and time resource, resulting in high costs for predicting organic chemical reaction products.

### SUMMARY

Embodiments of this application provide a training method and an application method for a reaction product prediction model, an apparatus, and a device, to implement automatic annotation on data of a sample reaction data set without relying on manual annotation, thereby reducing costs of reaction product prediction tasks, and improving accuracy of predicting a reaction product by a reaction product prediction model.

An aspect of embodiments of this application provides a training method for a reaction product prediction model, performed by a computer device, the method including: performing vector conversion on each reaction array in a sample reaction data set through an encoding network in a reaction product prediction model, to obtain a sample reactant vector and a sample reaction product vector, the sample reaction data set comprising a plurality of reaction arrays, each reaction array comprising a sample reactant and a sample reaction product; constructing a positive sample reactant set and a negative sample reactant set through a first auxiliary network according to the sample reactant vector; determining a reaction predicted loss value based on the positive sample reactant set and the negative sample reactant set; constructing a positive sample reaction group set and a negative sample reaction group set through a second auxiliary network according to the sample reactant vector and the sample reaction product vector; determining a reaction relationship predicted loss value based on the positive sample reaction group set and the negative sample reaction group set; determining, through a third auxiliary network according to the sample reactant vector and the sample reaction product vector, an atom label and a predicted probability value of an atom in the sample reactant being present in a main product; determining an atom predicted loss value based on the predicted probability value and the atom label; and training the reaction product prediction model based on the reaction predicted loss value, the reaction relationship predicted loss value, and the atom predicted loss value, to obtain a target reaction product prediction model.

Another aspect of this application provides an application method for a reaction product prediction model, performed by a computer device, the method including: inputting a to-be-measured reactant into a target reaction product prediction model, and outputting a predicted change probability of an adjacency matrix through the target reaction product prediction model; determining a predicted change amount of the adjacency matrix based on the predicted change probability of the adjacency matrix; and determining a target reaction product based on the predicted change amount of the adjacency matrix and an adjacency matrix of the to-be-measured reactant.

Another aspect of this application provides a non-transitory computer-readable storage medium, including instructions, causing, when run on a computer, the computer to perform the method according to the foregoing aspects.

It can be learned from the foregoing technical solutions that; the embodiments of this application have the following beneficial effects:

After the sample reactant vector and the sample reaction product vector are obtained, the positive sample reactant set and the negative sample reactant set are constructed through the first auxiliary network. The reaction predicted loss value is determined based on the positive sample reactant set and the negative sample reactant set, and the positive sample reaction group set and the negative sample reaction group set are constructed through the second auxiliary network. The reaction relationship predicted loss value is determined based on the positive sample reaction group set and the negative sample reaction group set. The atom label and the predicted probability value of the atom in the sample reactant being present in the main product are obtained through the third auxiliary network, and the atom predicted loss value is determined based on the predicted probability value and the atom label. Then, the reaction product prediction model may be trained based on the reaction predicted loss value, the reaction relationship predicted loss value, and the atom predicted loss value, to obtain the target reaction product prediction model. In the foregoing method, based on the first auxiliary network, the second auxiliary network, and the third auxiliary network that have self-supervision properties, the positive and negative sample sets and the atom label are mined and constructed according to data characteristics of the sample reaction data set, to implement automatic annotation on data of the sample reaction data set without relying on manual annotation, to reduce costs of training the reaction product prediction model. Moreover, the first auxiliary network can help the reaction product prediction model better learn of a distance relationship between reactants, so that the reaction product prediction model has a capability of predicting whether reactants can react. The second auxiliary network can help the reaction product prediction model better learn of a distance relationship between a reactant and a product, so that reaction product prediction model has a capability of predicting a correspondence between a reactant and a product. The third auxiliary network can help the reaction product prediction model better learn of a change of a key relationship between atoms in a reactant during reaction, so that the reaction product prediction model has a capability of predicting whether atoms are still present in a main product after the reaction. Assisted learning based on the auxiliary networks improves accuracy of predicting a reaction product by the reaction product prediction model.

### BRIEF DESCRIPTION OF THE DRAWINGS

FIG. 1 is a schematic architectural diagram of a reaction data control system according to an embodiment of this application.

FIG. 2 is a flowchart of an embodiment of a training method for a reaction product prediction model according to an embodiment of this application.

FIG. 3 is a flowchart of another embodiment of a training method for a reaction product prediction model according to an embodiment of this application.

FIG. 4 is a flowchart of another embodiment of a training method for a reaction product prediction model according to an embodiment of this application.

FIG. 5 is a flowchart of another embodiment of a training method for a reaction product prediction model according to an embodiment of this application.

FIG. 6 is a flowchart of another embodiment of a training method for a reaction product prediction model according to an embodiment of this application.

FIG. 7 is a flowchart of another embodiment of a training method for a reaction product prediction model according to an embodiment of this application.

FIG. 8 is a flowchart of another embodiment of a training method for a reaction product prediction model according to an embodiment of this application.

FIG. 9 is a flowchart of another embodiment of a training method for a reaction product prediction model according to an embodiment of this application.

FIG. 10 is a flowchart of another embodiment of a training method for a reaction product prediction model according to an embodiment of this application.

FIG. 11 is a flowchart of another embodiment of a training method for a reaction product prediction model according to an embodiment of this application.

FIG. 12 is a flowchart of another embodiment of a training method for a reaction product prediction model according to an embodiment of this application.

FIG. 13 is a flowchart of an embodiment of an application method for a reaction product prediction model according to an embodiment of this application.

FIG. 14 is a flowchart of another embodiment of an application method for a reaction product prediction model according to an embodiment of this application.

FIG. 15 is a schematic diagram of an auxiliary network in a training method for a reaction product prediction model according to an embodiment of this application.

FIG. 16 is a schematic diagram of a framework of a reaction product prediction model in a training method for a reaction product prediction model according to an embodiment of this application.

FIG. 17 is a schematic diagram of an embodiment of a training apparatus for a reaction product prediction model according to an embodiment of this application.

FIG. 18 is a schematic diagram of an embodiment of an application apparatus for a reaction product prediction model according to an embodiment of this application.

FIG. 19 is a schematic diagram of an embodiment of a computer device according to an embodiment of this application.

### DESCRIPTION OF EMBODIMENTS

For convenience of understanding, terms or concepts included in the embodiments of this application are explained first.

### 1. Transformer

A transformer includes an encoder and a decoder, and can use a self-attention mechanism without using a sequential structure of a recurrent neural network (RNN) and a long short-term memory (LSTM), to enable a model to be trained in parallel and possess global information.

### 2. Contrastive Learning

Contrastive learning is a self-supervised learning method configured to learn common features of a data set by making a model learn, without labels, which data points are similar or different.

### 3. Graph Neural Network

A graph neural network includes a graph convolutional network, a graph attention network, a graph auto-encoder, a graph generative network, a graph spatial-temporal network, and the like, and compared with a fully-connected layer (MLP) that is the most basic in a neural network and that is a feature matrix multiplied by a weight matrix, the graph neural network has an additional adjacency matrix.

### 4. Auxiliary Task

An auxiliary task is an important method for helping learning of a main task in reinforcement learning. During the learning of the main task, due to sparsity of rewards and difficulty of the task, an auxiliary task may be used to help learn feature representations.

It may be understood that, in a specific implementation of this application, related data, such as a sample reaction data set, may be involved. In a case that the foregoing embodiment of this application is applied to a specific product or technology, permission or an approval from a user needs to be obtained. In addition, collection, use, and processing of the related data need to comply with relevant laws, regulations, and standards of relevant countries and regions.

It is to be understood that the training method for a reaction product prediction model and the application method for a reaction product prediction model provided in this application may be applied to various scenarios, including, but not limited to, artificial intelligence, cloud technology, computational chemistry, pharmaceuticals, and the like, for optimizing a reaction product prediction task by training a powerful reaction product prediction model, for application to scenarios such as drug retrosynthesis authentication, scientific law mining, and pharmaceutical research and development.

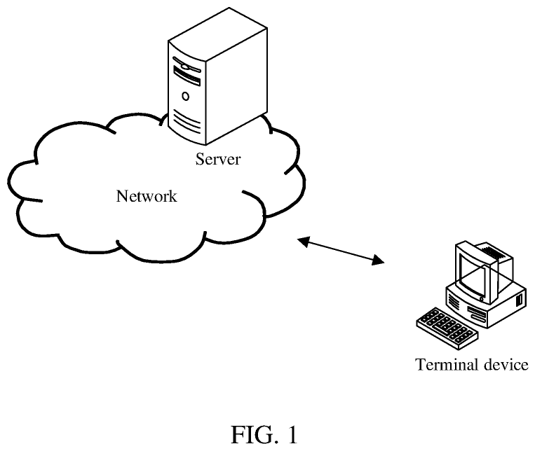

The training method for a reaction product prediction model provided in this application may be applied to a reaction data control system shown in FIG. 1. FIG. 1 is a schematic architectural diagram of a reaction data control system according to an embodiment of this application. As shown in FIG. 1, a server obtains a sample reactant vector and a sample reaction product vector based on a sample reaction data set provided by a terminal device, constructs a positive sample reactant set and a negative sample reactant set through a first auxiliary network, determines a reaction predicted loss value based on a positive sample reactant set and a negative sample reactant set, constructs a positive sample reaction group set and a negative sample reaction group set through a second auxiliary network, determines a reaction relationship predicted loss value based on the positive sample reaction group set and the negative sample reaction group set, obtains an atom label and a predicted probability value of an atom in a sample reactant being present in a main product through a third auxiliary network, and determines an atom predicted loss value based on the predicted probability value and the atom label, and then, may train a reaction product prediction model based on the reaction predicted loss value, the reaction relationship predicted loss value, and the atom predicted loss value, to obtain a target reaction product prediction model. In the foregoing method, based on the first auxiliary network, the second auxiliary network, and the third auxiliary network that have self-supervision properties, the positive and negative sample sets and the atom label can be mined and constructed according to data characteristics of the sample reaction data set, to implement automatic annotation on data of the sample reaction data set without relying on manual annotation, to reduce costs of training the reaction product prediction model. The first auxiliary network can help the reaction product prediction model better learn of a distance relationship between reactants, so that the reaction product prediction model has a capability of predicting whether reactants can react. The second auxiliary network can help the reaction product prediction model better learn of a distance relationship between a reactant and a product, so that reaction product prediction model has a capability of predicting a correspondence between a reactant and a product. The third auxiliary network can help the reaction product prediction model better learn of a change of a key relationship between atoms in a reactant during reaction, so that reaction product prediction model has a capability of predicting whether atoms are present in a main product after the reaction. Assisted learning based on the auxiliary networks can improves accuracy of predicting a reaction product by the reaction product prediction model.

It may be understood that FIG. 1 shows only one type of terminal device. In one embodiment, a plurality of types of terminal devices may participate in data processing. The terminal device includes, but is not limited to, a mobile phone, a computer, an intelligent voice interaction device, an intelligent household appliance, an in-vehicle terminal, and the like. A specific quantity and a specific type depend on one embodiment, and are not specifically limited herein. In addition, FIG. 1 shows only one server. However, there may be a plurality of servers participating in one embodiment, particularly, in a multi-model training interaction scenario. A quantity of servers depends on one embodiment, and is not specifically limited herein.

In this embodiment, the server may be an independent physical server, or may be a server cluster or a distributed system formed by a plurality of physical servers, or may be a cloud server that provides basic cloud computing services such as a cloud service, a cloud database, cloud computing, a cloud function, cloud storage, a network service, cloud communication, a middleware service, a domain name service, a security service, a content delivery network (CDN), big data, and an AI platform. The terminal device may be connected to the server directly or indirectly in a wired or wireless communication method, and the terminal device may be connected to the server to form a blockchain network, which is not limited this application.

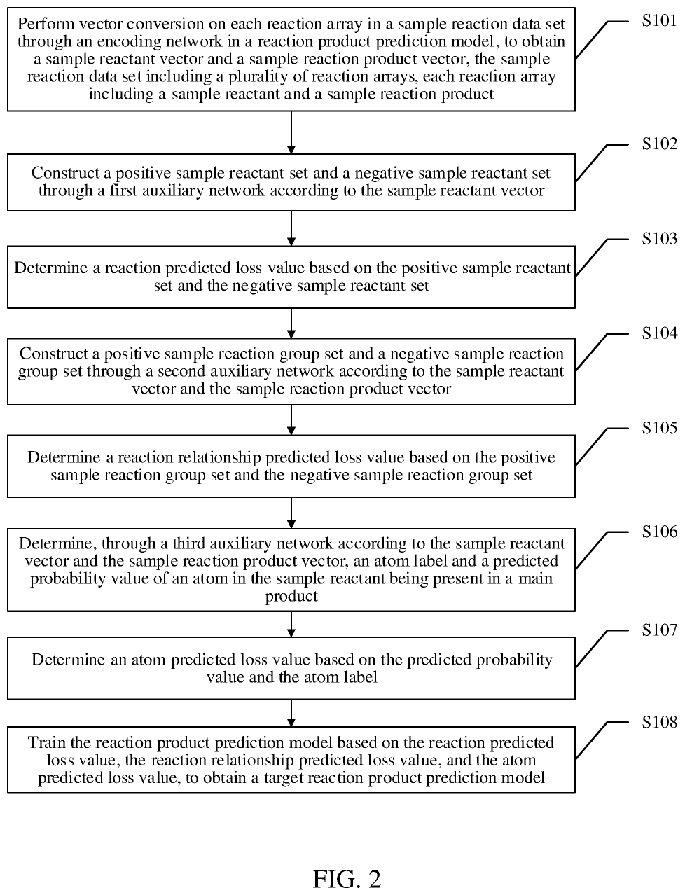

The training method for a reaction product prediction model according to this application is described below with reference to the foregoing descriptions. The method may be performed by a computer device. The computer device may be a server or a terminal device. Referring to FIG. 2, an embodiment of the training method for a reaction product prediction model includes the following steps:

Step S101: Perform vector conversion on each reaction array in a sample reaction data set through an encoding network in a reaction product prediction model, to obtain a sample reactant vector and a sample reaction product vector, the sample reaction data set including a plurality of reaction arrays, each reaction array including a sample reactant and a sample reaction product.

It may be understood that the sample reaction data set includes a plurality of reaction arrays. Each reaction array may be represented as (GT, G^{P}). Each reaction array is configured to represent one piece of organic chemical reaction data R₁+R₂+ . . . →P₁+P₂+. . . , G^{r} represents a series of sample reactants, and G^{p} represents a series of products, that is, sample reaction products.

In a molecular graph G=(V, E), V represents an atom set, and a size of the set is a quantity |V|=N of atoms. Each atom v E V is associated with an atom feature, including a type of the atom, chargeability, aromaticity, and the like. E represents a set of edges, and each edge is associated with a bond type, including a single bond, a double bond, a triple bond, an aromatic bond, and the like. A target of organic chemical reaction prediction is to predict a product given a reactant.

Further, due to the atom-mapping principle in organic chemical reactions, atoms in a reactant have a one-to-one correspondence with atoms in a product. Therefore, main changes before and after a reaction are changes of connections between atoms, that is, a change of an adjacency matrix A, and involved atoms do not change. Therefore, to predict a product (that is, a reaction product), only its corresponding adjacency matrix needs to be predicted, so that the structure of the entire product can be restored. The adjacency matrix used in this embodiment is different from a common adjacency matrix. Further, because it is easier to predict a change, that is, AA, of the adjacency matrix A than directly predicting A of the product, in this embodiment, an organic chemical reaction product prediction problem may be transformed into modeling a probability distribution P(ΔΔ|G^{r}).

To subsequently better model the probability distribution P(ΔA|G^{r}), a sample reaction data set is obtained first, and the sample reaction data set is input into a reaction product prediction model. Vector conversion is performed on each reaction array in the sample reaction data set through an encoding network of the reaction product prediction model, to obtain a sample reactant vector and a sample reaction product vector corresponding to the reaction array, that is, a sample reactant vector corresponding to a sample reactant in the reaction array and a sample reaction product vector corresponding to a sample reaction product in the reaction array.

The reaction product prediction model may be specifically expressed as a VAE architecture satisfying electron transfer conservation shown in FIG. 16, and may also be expressed as another model, for example, a flow-based model, maximum likelihood training, a deep VAE, or the like, which is not specifically limited herein.

Further, if the VAE architecture satisfying electron transfer conservation shown in FIG. 16 is used as the reaction product prediction model, vector conversion may be performed on each reaction array in the sample reaction data set through a graph neural network GNN and a transformer shown in FIG. 16, to obtain a sample reactant vector and a sample reaction product vector.

Step S102: Construct a positive sample reactant set and a negative sample reactant set through a first auxiliary network according to the sample reactant vector.

In this embodiment, because in the modeling process, all predictions for a reaction product assume that a given reactant can react, but in one embodiment, not all reactants can react, a correct reaction product prediction model is to have a capability of distinguishing whether a reactant can react. Moreover, because in embedding space, molecules that cannot react are far apart, and molecules that can react are close to each other, in this embodiment, a positive sample reactant set and a negative sample reactant set are constructed by constructing an auxiliary task for predicting whether a reactant can react, that is, a first auxiliary network, to help the reaction product prediction model learn a distance between molecules, to learn to distinguish whether a reactant has a capability of reacting, thereby enhancing the generalization capability of molecule representation. Therefore, after the sample reactant vector is obtained, the sample reactant vector is input into the first auxiliary network, and the positive sample reactant set, and the negative sample reactant set are constructed through the first auxiliary network.

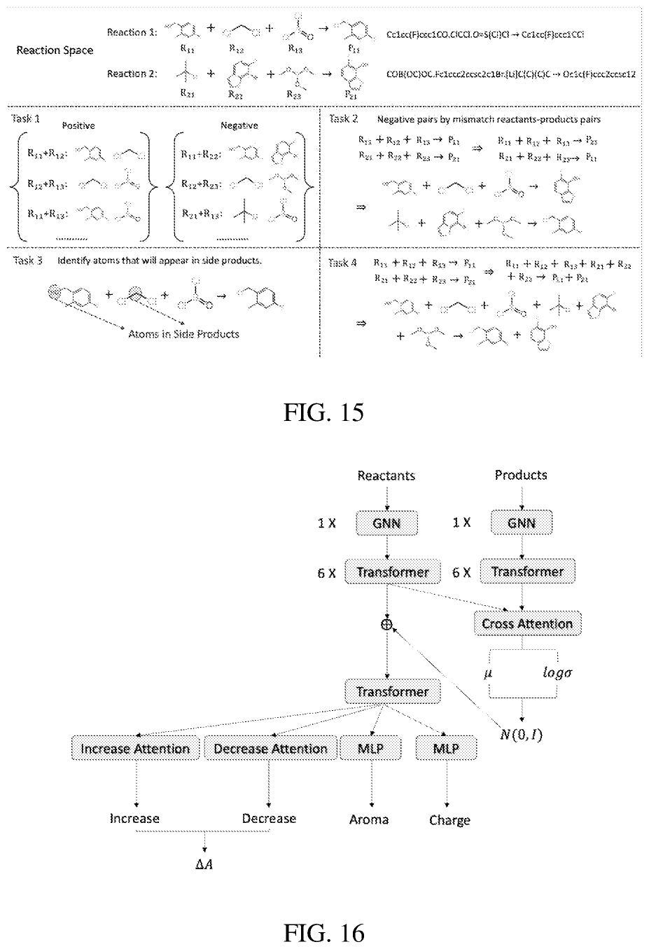

Specifically, the sample reactant vector is input into a task Task1, that is, the first auxiliary network, shown in FIG. 15, sample reactant vectors corresponding to any two sample reactants are sampled from a same reaction array, for example, R₁₁+R₁₂+R₁₃→P₁₁, as a positive sample reactant combination, for example, R₁₁+R₁₂, R₁₁+R₁₃, and R₁₂+R₁₃, so that the positive sample reactant combination may be added to the positive sample reactant set positive.

Further, sample reactant vectors corresponding to any two sample reactants are sampled from different reaction arrays, for example, R₁₁+R₁₂+R₁₃→P₁₁ and R₂₁+R₂₂+R₂₃→P₂₁, as a negative sample reactant combination, for example, R₁₁+R₂₂, R₁₂+R₂₃, and R₂₁+R₁₃, so that the negative sample reactant combination may be added to the negative sample reactant set negative.

Step S103: Determine a reaction predicted loss value based on the positive sample reactant set and the negative sample reactant set.

In this embodiment, the reaction predicted loss value is configured to represent the reaction product prediction model's capability of distinguishing whether a reactant can react. That is, the reaction predicted loss value is data configured to represent the reaction product prediction model's prediction accuracy for a reaction possibility. The reaction possibility refers to whether a reactant input into the reaction product prediction model can react.

Specifically, after the positive sample reactant set and the negative sample reactant set are obtained, the reaction predicted loss value may be calculated using the following loss function formula (1):

$${L}_{A} = {\sum }_{( i , j ) ∈ {D}_{pos}} max ( {‖ {h}_{i} - {h}_{j} ‖}_{2} - ε , 0 ) + {\sum }_{( i , j ) ∈ {D}_{neg}} max ( γ - {‖ {h}_{i} - {h}_{j} ‖}_{2} , 0 ) \qquad ( 1 )$$

where Dₚₒₛ represents the positive sample reactant set, D_{neg} represents negative sample reactant set, (i, j) are configured to respectively represent a positive sample reactant combination or a negative sample reactant combination sampled from the corresponding sample reactant set, ε and γ are two margin hyperparameters, hᵢ and hⱼ are respectively sample reactant vector embeddings of sample reactants i and j. It may be understood that a first term of the forward loss function formula (1) can be configured to reduce an embedding distance between positive sample molecules, and a second term thereof can be configured to increase an embedding distance between negative sample molecules.

Step S104: Construct a positive sample reaction group set and a negative sample reaction group set through a second auxiliary network according to the sample reactant vector and the sample reaction product vector.

In this embodiment, because a ranking of a candidate product is a piece of important information, allowing the reaction product prediction model to receive possibility scores about different candidate products, an auxiliary task configured to predict whether a reactant and a product have a correct correspondence, that is, a second auxiliary network is constructed to help the reaction product prediction model learn of a relationship between a reactant and a reaction product, to learn of a capability of distinguishing whether the reactant and the reaction product have a correct correspondence, thereby enhancing the generalization capability of molecule representation. Therefore, after the sample reactant vector and the sample reaction product vector are obtained, the sample reactant vector and the sample reaction product vector are input into the second auxiliary network, and the positive sample reaction group set and the negative sample reaction group set are constructed through the second auxiliary network.

Specifically, the sample reactant vector and the sample reaction product vector are input into Task2, that is, the second auxiliary network, shown in FIG. 15. Sample reactant vectors and sample reaction product vectors corresponding to all reaction arrays, where, for example, in a reaction array R₁₁+R₁₂+R₁₃→P₁₁, reactants R₁₁+R₁₂+R₁₃ have a correct correspondence with a reaction product P₁₁, may be used as a positive sample reaction group set.

Further, a negative sample reaction group set may be constructed by mismatching a reactant and a reaction product, to make a matching score between a reactant and a reaction product having an incorrect correspondence lower. For example, in a reaction array R₁₁+R₁₂+R₁₃→P₁₁, reactants R₁₁+R₁₂+R₁₃ and a reaction product P₁₁ have a correct correspondence, and in another reaction array R₂₁+R₂₂+R₂₃→P₂₁, reactants R₂₁+R₂₂+R₂₃ and a reaction product P₂₁ have a correct correspondence. A negative sample reaction group set including incorrect correspondences between reactants and reaction products, such as R₁₁+R₁₂+R₁₃→P₂₁ and R₂₁+R₂₂+R₂₃→P₁₁, may be obtained by mismatching the reactants and the reaction products.

Step S105: Determine a reaction relationship predicted loss value based on the positive sample reaction group set and the negative sample reaction group set.

In this embodiment, the reaction relationship predicted loss value is configured to represent the reaction product prediction model's capability of distinguishing whether a reactant has a correct correspondence with a reaction product. That is, the reaction relationship predicted loss value is data configured to represent the reaction product prediction model's prediction accuracy for a reaction relationship. The reaction relationship refers to a correspondence between a reactant and a reaction product. The reaction relationship predicted loss value herein and the reaction predicted loss value mentioned in step 103 above are two parallel loss values. Both can be configured in the subsequent steps to train the reaction product prediction model. However, there is no necessary association between the two, and the two represent different information. The reaction predicted loss value is configured to represent the reaction product prediction model's capability of identifying whether a reaction can occur between reactants. The reaction relationship predicted loss value is configured to represent the reaction product prediction model's capability of identifying whether there is a correct correspondence between a reactant and a reaction product.

Specifically, after the positive sample reaction group set and the negative sample reaction group set are obtained, the reaction relationship predicted loss value may be calculated using the following loss function formula (2):

$${L}_{B} = \frac{1}{\vert B \vert} {\sum }_{i} max ( {‖ {G}_{θ} ( {\{ {h}_{r} \}}_{r ∈ {R}_{i}} ) - {G}_{φ} ( {\{ {h}_{p} \}}_{p ∈ {P}_{i}} ) ‖}_{2} - ε , 0 ) + \frac{1}{\vert B \vert ( \vert B \vert - 1 )} {\sum }_{i \neq j} max ( γ - {‖ {G}_{θ} ( {\{ {h}_{r} \}}_{r ∈ {R}_{i}} ) - {G}_{φ} ( {\{ {h}_{p} \}}_{p ∈ {P}_{j}} ) ‖}_{2} , 0 ) \qquad ( 2 )$$

where G_{θ} is a reactant network, G_{φ} is a reaction product network, G_{θ} and G_{φ} have different parameters of the same architecture, and both the networks satisfy permutation invariance. hᵣ is a feature vector embedding corresponding to a reactant atom, hₚ is a feature vector embedding corresponding to a reaction product atom, are ε and γ are two margin hyperparameters. A first term of the loss function is configured to reduce a distance between a sample reactant vector and a sample reaction product vector of the same reaction array, that is, the positive sample reaction group set, and a second term thereof is configured to push a sample reactant vector and a sample reaction product vector of a mismatched reaction array, that is, the negative sample reaction group set, away from each other. It may be understood that, because a task may be constructed online during model training, the size of a sample of the objective function may be set to B.

Step S106: Determine, through a third auxiliary network according to the sample reactant vector and the sample reaction product vector, an atom label and a predicted probability value of an atom in the sample reactant being present in a main product.

In this embodiment, because side products are often ignored in reaction data in massive data sets, for example, the disclosed data set USPTO-480K, it is easy to increase a prediction error of a reaction product. Therefore, in this embodiment, a third auxiliary network configured to predict whether an atom is present in a main product may be added to the modeling process, to supplement information about some side products to some extent, thereby improving the capability of predicting the reaction product. Therefore, after the sample reactant vector and the sample reaction product vector are obtained, the sample reactant vector and the sample reaction product vector are input into the third auxiliary network, and an atom label and a predicted probability value of an atom in the sample reactant being present in a main product are determined through the third auxiliary network.

Specifically, the sample reactant vector and the sample reaction product vector are inputted to a task Task3, that is, the third auxiliary network, shown in FIG. 15. Prediction is performed on a sample reactant through the third auxiliary network, to obtain a predicted probability value of an atom in the sample reactant being present in a main product. In addition, an atom in the sample reactant is compared with an atom in the sample reaction product based on the sample reactant vector and the sample reaction product vector, to obtain an atom comparison result, and an atom label is determined based on the atom comparison result.

Step S107: Determine an atom predicted loss value based on the predicted probability value and the atom label.

In this embodiment, the atom predicted loss value is configured to represent the capability of the reaction product prediction model to distinguish whether an atom is still in the main product after the reaction.

Specifically, after the predicted probability value and the atom label are obtained, the atom predicted loss value may be calculated using the following loss function formula (3):

$${L}_{C} = \frac{1}{\vert B \vert} {\sum }_{i} {\sum }_{j} {y}_{j} log {s}_{j} + ( 1 - {y}_{j} ) log ( 1 - {s}_{j} ) \qquad ( 3 )$$

where yⱼ∈{0,1} represents an atom label of an atom j, indicating whether the atom is in the main product after the reaction, and sⱼ∈[0,1] represents a predicted probability value of the based on an atom embedding.

Step S108: Train the reaction product prediction model based on the reaction predicted loss value, the reaction relationship predicted loss value, and the atom predicted loss value, to obtain a target reaction product prediction model.

Specifically, when the reaction predicted loss value, the reaction relationship predicted loss value, and the atom predicted loss value are obtained, a multi-task learning training method may be used to directly add objective functions, such as the reaction predicted loss value, the reaction relationship predicted loss value, and the atom predicted loss value, to a total objective function in a weighted method, and then, parameter adjustment is performed on the reaction product prediction model based on the total objective function value. Specifically, parameter adjustment may be performed using an iterative method of backpropagation or another method, which is not specifically limited herein, to obtain the target reaction product prediction model.

According to another aspect, in this embodiment, the first auxiliary network, the second auxiliary network, and the third auxiliary network may be pre-trained respectively based on the reaction predicted loss value, the reaction relationship predicted loss value, and the atom predicted loss value by using a pre-training policy, and then, the pre-trained networks are migrated to the reaction product prediction model to obtain the target reaction product prediction model.

In this embodiment, a training method for a reaction product prediction model is provided. In the foregoing method, based on the first auxiliary network, the second auxiliary network, and the third auxiliary network that have self-supervision properties, the positive and negative sample sets and the atom label can be mined and constructed according to data characteristics of the sample reaction data set, to implement automatic annotation on data of the sample reaction data set without relying on manual annotation, to reduce costs of training the reaction product prediction model. The first auxiliary network can help the reaction product prediction model better learn of a distance relationship between reactants, so that the reaction product prediction model has a capability of predicting whether reactants can react. The second auxiliary network can help the reaction product prediction model better learn of a distance relationship between a reactant and a product, so that reaction product prediction model has a capability of predicting a correspondence between a reactant and a product. The third auxiliary network can help the reaction product prediction model better learn of a change of a key relationship between atoms in a reactant during reaction, so that reaction product prediction model has a capability of predicting whether atoms are present in a main product after the reaction. Assisted learning based on the auxiliary networks can improve accuracy of predicting a reaction product by the reaction product prediction model. Accordingly, the target reaction product prediction model trained may be used as an effective verification tool for drug retrosynthesis, to improve the efficiency of research on new drug synthesis paths. In addition, the target reaction product prediction model may also predict a reliable candidate product when no known template can be used, and can predict a new reaction, thereby greatly improving the efficiency of researching and development of new drugs.

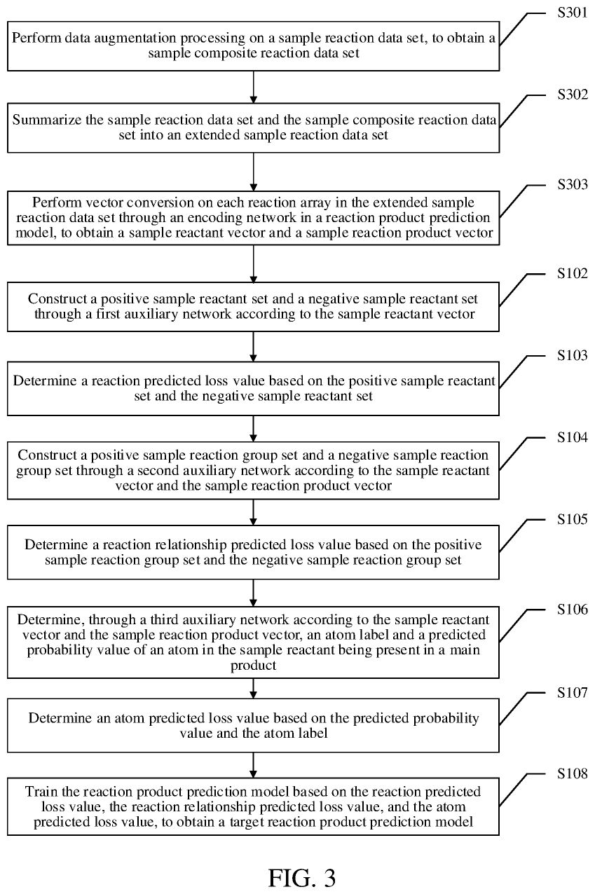

In some embodiments, based on the embodiment corresponding to FIG. 2, in another embodiment of the training method for a reaction product prediction model provided in this application, as shown in FIG. 3, before the performing vector conversion on each reaction array in a sample reaction data set through an encoding network in a reaction product prediction model, to obtain a sample reactant vector and a sample reaction product vector in step S101, the method further includes: step S301 to step S302; and step S101 includes step S303.

Step S301: Perform data augmentation processing on the sample reaction data set, to obtain a sample composite reaction data set.

Step S302: Summarize the sample reaction data set and the sample composite reaction data set into an extended sample reaction data set.

Step S303: Perform vector conversion on each reaction array in the extended sample reaction data set through the encoding network in the reaction product prediction model, to obtain the sample reactant vector and the sample reaction product vector.

In this embodiment, although a common reaction product prediction model can predict a simple chemical reaction with, for example, one reactant or two or three reactants, it is difficult to predict a chemical reaction with more reactants or a complex chemical reaction. Based on this, in this embodiment, data augmentation is performed on the sample reaction data set during the modeling, to the sample composite reaction data set, and the sample reaction data set and the sample composite reaction data set are summarized into the extended sample reaction data set; and then, the extended sample reaction data set is inputted to the reaction product prediction model, and vector conversion is performed on each reaction array in the extended sample reaction data set through the encoding network of the reaction product prediction model, to obtain the sample reactant vector and the sample reaction product vector. Accordingly, not only the sample reaction data set can be expanded, but also the complexity of the sample reaction data set can be enhanced to some extent, to help the reaction product prediction model learn of the capability of predicting a complex chemical reaction, thereby improving the accuracy of predicting a reaction product to some extent.

Specifically, as indicated by a task Task4 shown in FIG. 15, data augmentation processing is performed on the sample reaction data set. Specifically, two reaction arrays may be randomly selected from the sample reaction data set, sample reactants in the two selected reaction arrays are combined, to obtain a sample composite reactant, sample reaction products in the two selected reaction arrays are combined to obtain a sample composite reaction product, and then, a composite reaction array is obtained based on the sample composite reactant and the sample composite reaction product, and a sample composite reaction data set is constructed based on the composite reaction array. Alternatively, another augmentation method may be adopted, which is not specifically limited herein. For example, reactants R₁₁+R₁₂+R₁₃ and a reaction product P₁₁ in one reaction array R₁₁+R₁₂+R₁₃→P₁₁ are combined with reactants R₂₁+R₂₂+R₂₃ and a reaction product P₂₁ in another reaction array R₂₁+R₂₂+R₂₃→P₂₁, to obtain a composite reaction array, for example, R₁₁+R₁₂+R₁₃+R₂₁+R₂₂+R₂₃→P₁₁+P₂₁.

Further, the obtained sample reaction data set and the obtained sample composite reaction data set are summarized into an extended sample reaction data set, to facilitate subsequently utilizing the extended sample reaction data set to train the reaction product prediction model, to help the reaction product prediction model to learn of the capability of predicting a complex chemical reaction. That is, the extended sample reaction data set is input into the reaction product prediction model, and vector conversion is performed on each reaction array in the extended sample reaction data set through the encoding network of the reaction product prediction model to obtain the sample reactant vector and the sample reaction product vector, so that the reaction product prediction model can be trained based on the sample reactant vector and the sample reaction product vector by combining the first auxiliary network, the second auxiliary network, and the third auxiliary network.

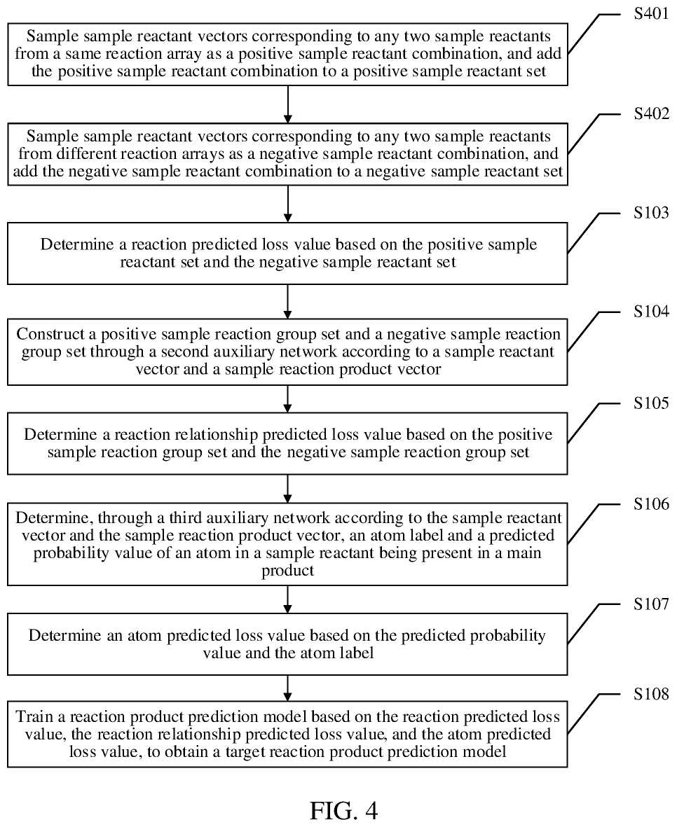

In some embodiments, based on the embodiment corresponding to FIG. 2 or FIG. 3, in another embodiment of the training method for a reaction product prediction model provided in this application, as shown in FIG. 4, the constructing a positive sample reactant set and a negative sample reactant set through a first auxiliary network according to the sample reactant vector in step S102 includes the following steps:

Step S401: Sample the sample reactant vectors corresponding to any two sample reactants from the same reaction array as a positive sample reactant combination, and add the positive sample reactant combination to the positive sample reactant set.

Step S402: Sample the sample reactant vectors corresponding to any two sample reactants from different reaction arrays as a negative sample reactant combination, and add the negative sample reactant combination to the negative sample reactant set.

Specifically, in embedding space, molecules that cannot react are far apart, and molecules that can react are close to each other. Therefore, after a sample reactant vector corresponding to each reaction array is obtained, according to chemical reaction data, for example, G₁^{R}^{ᵢ}+G₂^{R}^{ᵢ}+ . . . →P₁^{P}^{ᵢ}+P₂^{P}^{ᵢ}+ . . . , where G₁^{R}^{ᵢ}, G₂^{R}^{ᵢ}, and the Like are Configured to Represent sample reactants, P₁^{P}^{ᵢ}, P₂^{P}^{ᵢ}, and the like are configured to represent sample reaction products, reactant combination data is constructed by combining reactants pairwise.

Further, as shown in FIG. 15, for convenience of expression, in this embodiment, given a batch of reaction data B, two spaces are constructed, where one is a positive sample space, that is, the positive sample reactant set, and the other is a negative sample space, that is, the negative sample reactant set. The sample reactant vector is input into the task Task1 shown in FIG. 15, that is, the first auxiliary network. A positive sample reactant set Dₚₒₛ is constructed according to reaction data given in chemical data B. That is, sample reactant vectors corresponding to any two sample reactants in the same reaction array are combined. Assuming that each reaction has R reactants, R(R−1) positive sample reactant combinations can be obtained. A negative sample reactant set D_{neg} may be obtained by combining sample reactants from different reaction arrays. That is, sample reactant vectors corresponding to any two sample reactants sampled from different reaction arrays are combined.

For example, as shown in FIG. 15, sample reactant vectors corresponding to any two sample reactants are sampled from a same reaction array, for example, R₁₁+R₁₂+R₁₃→P₁₁, as a positive sample reactant combination, for example, R₁₁+R₁₂, R₁₁+R₁₃, and R₁₂+R₁₃, so that a positive sample reactant set positive can be obtained through summarization.

Further, any two sample reactant vectors are sampled from different reaction arrays, for example, R₁₁+R₁₂+R₁₃→P₁₁ and R₂₁+R₂₂+R₂₃→P₂₁, as a negative sample reactant combination, for example, R₁₁+R₂₂, R₁₂+R₂₃, and R₂₁+R₁₃, so that a negative sample reactant set negative can be obtained through summarization.

Accordingly, by constructing the positive sample reactant set and the negative sample reactant set in the foregoing way, the first auxiliary network can better help the reaction product prediction model learn of a distance relationship between reactants, so that the reaction product prediction model can predict more accurately whether a reactant can react.

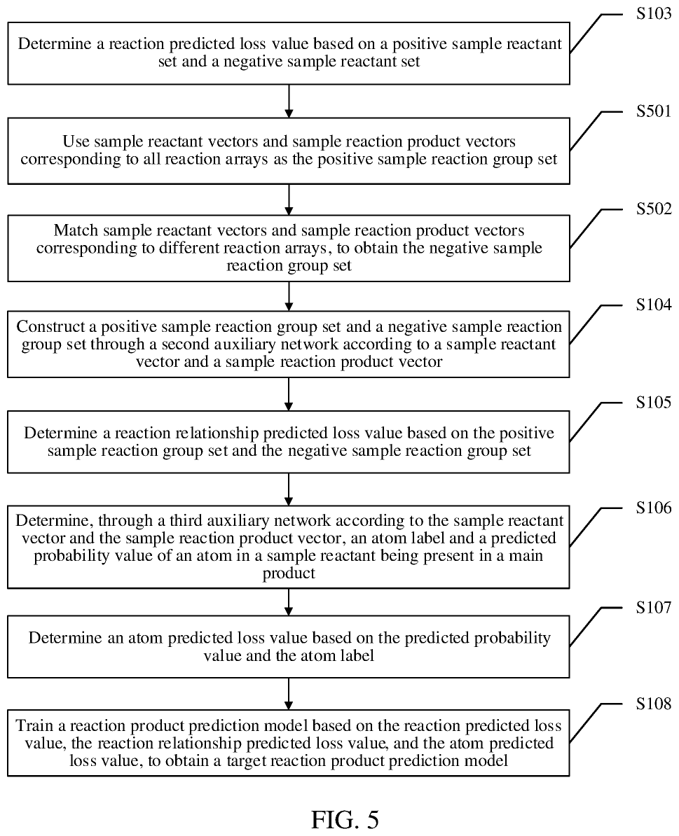

In some embodiments, based on the embodiment corresponding to FIG. 2 or FIG. 3, in another embodiment of the training method for a reaction product prediction model provided in this application, as shown in FIG. 5, the constructing a positive sample reaction group set and a negative sample reaction group set through a second auxiliary network according to the sample reactant vector and the sample reaction product vector in step S104 includes the following steps:

Step S501: Use the sample reactant vectors and the sample reaction product vectors corresponding to all the reaction arrays as the positive sample reaction group set.

Step S502: Match the sample reactant vectors and the sample reaction product vectors corresponding to different reaction arrays, to obtain the negative sample reaction group set.

Specifically, a batch of chemical reaction data B={R₁→P₁, R₂→P₂, . . . } may be given, and a given reactant, for example, R₁ and a reaction product P₁ are combined as a positive sample reaction group, that is, R₁→P₁. That is, chemical reaction data B may be used as a positive sample reaction group set. Specifically, the sample reactant vector and the sample reaction product vector are inputted to Task2, that is, the second auxiliary network, shown in FIG. 15. Sample reactant vectors and sample reaction product vectors corresponding to all reaction arrays, where, for example, in a reaction array R₁₁+R₁₂+R₁₃→P₁₁, reactants R₁₁+R₁₂+R₁₃ have a correct correspondence with a reaction product P₁₁, may be used as a positive sample reaction group in the positive sample reaction group set.

Further, the negative sample reaction group set is constructed through combinations of reactants and reaction products in mismatched chemical reaction data B={R₁→P₁, R₂→P₂, . . . }, and the sample reactant vector and the sample reaction product vector are mismatched, to obtain a negative sample reaction group, and make a matching score between a reactant and a reaction product having an incorrect correspondence lower. For example, as shown in FIG. 15, in a reaction array R₁₁+R₁₂+R₁₃→P₁₁, reactants R₁₁+R₁₂+R₁₃ and a reaction product P₁₁ have a correct correspondence, and in another reaction array R₂₁+R₂₂+R₂₃→P₂₁, reactants R₂₁+R₂₂+R₂₃ and a reaction product P₂₁ have a correct correspondence. A negative sample reaction group including incorrect correspondences between reactants and reaction products, such as R₁₁+R₁₂+R₁₃→P₂₁ and R₂₁+R₂₂+R₂₃→P₁₁, may be obtained by mismatching the reactants and the reaction products.

Accordingly, by constructing the positive sample reaction group set and the negative sample reaction group set in the foregoing way, the second auxiliary network can better help the reaction product prediction model learn a distance relationship between a reactant and a product, so that the reaction product prediction model can predict more accurately a correspondence between a reactant and a product.

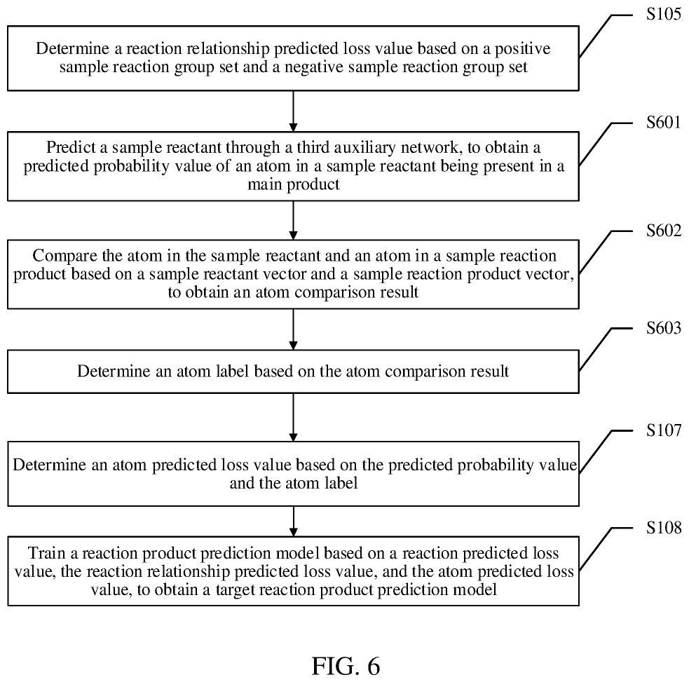

In some embodiments, based on the embodiment corresponding to FIG. 2 or FIG. 3, in another embodiment of the training method for a reaction product prediction model provided in this application, as shown in FIG. 6, the determining, through a third auxiliary network according to the sample reactant vector and the sample reaction product vector, an atom label and a predicted probability value of an atom in the sample reactant being present in a main product in step S106 includes the following steps:

Step S601: Predict the sample reactant through the third auxiliary network, to obtain the predicted probability value of the atom in the sample reactant being present in the main product.

Step S602: Compare the atom in the sample reactant and an atom in the sample reaction product based on the sample reactant vector and the sample reaction product vector, to obtain an atom comparison result.

Step S603: Determine the atom label based on the atom comparison result.

In this embodiment, because predicting whether an atom is present in a main product and predicting a reaction center are two tasks that are not equivalent to each other, by predicting the reaction center, only which bonds are broken can be learned of, and which one of two atoms connected by a bond is present in a side product cannot be learned of, the third auxiliary network configured to predict whether an atom is present in a main product allows the reaction product prediction model to learn of relative importance of the atom in the reaction. Therefore, prediction may be performed on a sample reactant through the third auxiliary network, to obtain a predicted probability value of an atom in the sample reactant being present in a main product. In addition, an atom in the sample reactant is compared with an atom in the sample reaction product based on the sample reactant vector and the sample reaction product vector, so that a corresponding atom comparison result can be obtained. Then, an atom label may be determined based on the atom comparison result.

Specifically, a sample reactant is predicted through the third auxiliary network, to obtain a predicted probability value of an atom in the sample reactant being present in a main product. Specifically, predicting whether the atom is present in the main product can be regarded as known information that is used as a masking matrix to mask side product information, so that the reaction product prediction model can only pay attention to information about the main product, and actually, the masking matrix is also content that the reaction product prediction model is to predict.

Further, a method of obtaining the atom label, which is a ground-truth label, may be based on sample reactant vector and sample reaction product vector. Atoms of the sample reactant are compared with atoms of the sample reaction product. An atom lost by the reactant, compared with the reaction product, is marked as 0, while an atom still remaining in the reaction product is marked as 1. Another labeling method may also be used, which is not specifically limited herein. Accordingly, in the foregoing method, the third auxiliary network can better help the reaction product prediction model learn of a change of a bond relationship between atoms of a reactant during a reaction, so that the reaction product prediction model can predict more accurately whether an atom is present in the main product after the reaction.

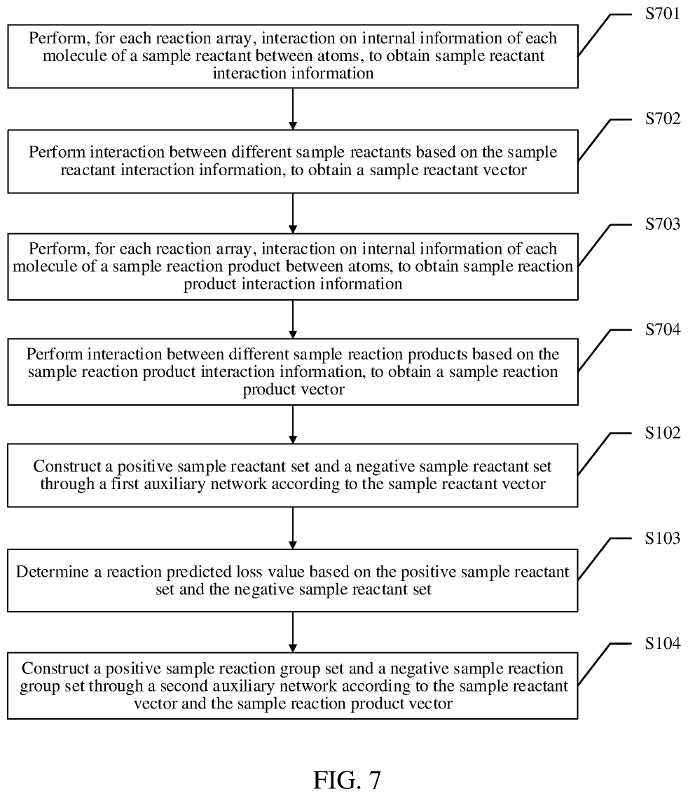

In some embodiments, based on the embodiment corresponding to FIG. 2 or FIG. 3, in another embodiment of the training method for a reaction product prediction model provided in this application, as shown in FIG. 7, the performing vector conversion on each reaction array in a sample reaction data set through an encoding network in a reaction product prediction model, to obtain a sample reactant vector and a sample reaction product vector in step S101 further includes the following steps:

Step S701: Perform, for each reaction array, interaction on internal information of each molecule of the sample reactant between atoms, to obtain sample reactant interaction information.

Step S702: Perform interaction between different sample reactants based on the sample reactant interaction information, to obtain the sample reactant vector.

Step S703: Perform, for each reaction array, interaction on internal information of each molecule of the sample reaction product between atoms, to obtain sample reaction product interaction information.

Step S704: Perform interaction between different sample reaction products based on the sample reaction product interaction information, to obtain the sample reaction product vector.

Specifically, when the VAE architecture satisfying electron transfer conservation shown in FIG. 16 is used as the reaction product prediction model, in this embodiment, the encoding network performing vector conversion on each reaction array in the sample reaction data set may be formed by combining a graph neural network GNN and a transformer.

The transformer includes an encoder and a decoder, and can use a self-attention mechanism without using a sequential structure of a recurrent neural network (RNN) and a long short-term memory L(STM), to enable a model to be trained in parallel and possess global information.

The graph neural network GNN includes a graph convolutional network, a graph attention network, a graph auto-encoder, a graph generative network, a graph spatial-temporal network, and the like, and compared with a fully-connected layer (MLP) that is the most basic in a neural network and that is a feature matrix multiplied by a weight matrix, the graph neural network has an additional adjacency matrix.

Therefore, interaction may first be performed on internal information of each molecule between atoms through the GNN by using the following formula (4). That is, for each reaction array, interaction is performed on internal information of each molecule of a sample reactant between atoms, to obtain sample reactant interaction information, and interaction is then performed on the information through the transformer between different reactants. Interaction is performed between different sample reactants based on the sample reactant interaction information, to obtain the sample reactant vector:

$${h}^{R} = Transformer ( GNN ( {G}^{R} ) ) \qquad ( 4 )$$

where h^{R} is configured to represent the sample reactant vector, and G^{R} is configured to represent the sample reactant.

Likewise, interaction may be first performed on internal information of each molecule between atoms through the GNN by using the following formula (5), that is, for each reaction array, interaction is performed on internal information of each molecule of a sample reaction product between atoms, to obtain sample reaction product interaction information, and interaction is then performed on the information through the transformer between different reaction products, that is, interaction is performed between different sample reaction products based on the sample reaction product interaction information, to obtain the sample reaction product vector:

$${h}^{P} = Transformer ( GNN ( {G}^{P} ) ) \qquad ( 5 )$$

where h^{P} is configured to represent the sample reaction product vector, and G^{P} is configured to represent the sample reaction product.

Accordingly, obtaining the sample reactant vector and the sample reaction product vector through the foregoing method can ensure that the obtained sample reactant vector and the obtained sample reaction product vector can more accurately reflect features of the corresponding sample reactant and the corresponding sample reaction product, and further, help the first auxiliary network, the second auxiliary network, and the third auxiliary network subsequently better learn of related capabilities based on the sample reactant vector and the sample reaction product vector, thereby improving the performance of the trained reaction product prediction model.

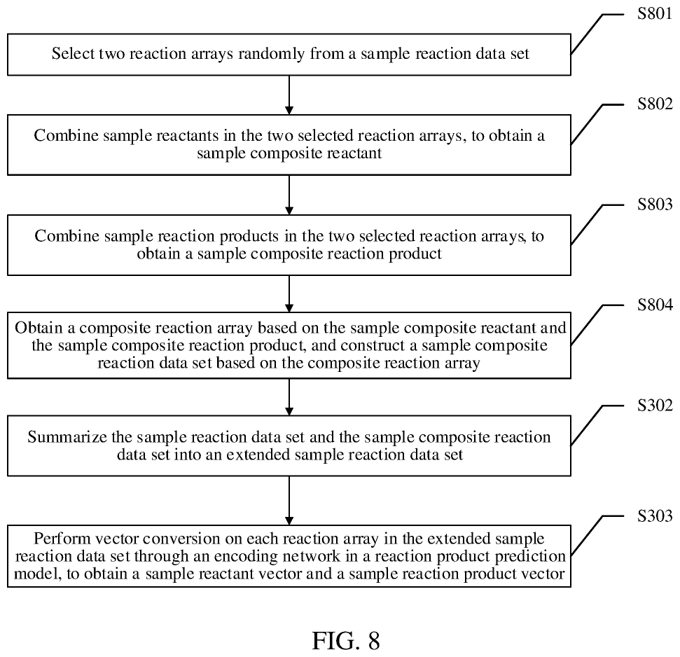

In some embodiments, based on the embodiment corresponding to FIG. 3, in another embodiment of the training method for a reaction product prediction model provided in this application, as shown in FIG. 8, the performing data augmentation processing on the sample reaction data set, to obtain a sample composite reaction data set, in step S301 includes the following steps:

Step S801: Select two reaction arrays randomly from the sample reaction data set.

Step S802: Combine sample reactants in the two selected reaction arrays, to obtain a sample composite reactant.

Step S803: Combine sample reaction products in the two selected reaction arrays, to obtain a sample composite reaction product.

Step S804: Obtain a composite reaction array based on the sample composite reactant and the sample composite reaction product, and construct the sample composite reaction data set based on the composite reaction array.

In this embodiment, performing data augmentation processing on the sample reaction data set may be randomly selecting two reaction arrays from the sample reaction data set, combining sample reactants in the two selected reaction arrays to obtain a sample composite reactant, in addition, combining sample reaction products in the two selected reaction arrays to obtain a sample composite reaction product, then, obtaining a composite reaction array based on the sample composite reactant and the sample composite reaction product, and constructing a sample composite reaction data set according to the composite reaction array. Although a completely accurate chemical reaction cannot be obtained through combination according to the data augmentation method, for example, between new reactant combinations, reactions may no longer be performed between A and B and between E and F, but between A and E and between B and F, or may be performed between other combinations, or even if reactions are still performed between A and B and between E and F, only intermediate products are obtained, and can be further reacted to obtain a final product, that is, new reactions obtained through random combination using the data augmentation method may be not accurate, the generalization capability of the reaction product prediction model can still be enhanced. In addition, there may be incorrect reaction data (for example, an intermediate product instead of a final product is given as a ground-truth product) in the commonly used USPTO-480K data. Therefore, inaccurate new reaction combinations obtained using the foregoing data augmentation method have small impact on the prediction accuracy of the reaction product prediction model, so that the sample composite reaction data set obtained after the data augmentation and the original sample reaction data set may be summarized to obtain through extension an extended sample reaction data set. The extended sample reaction data set is subsequently applied to parameter adjustment on the reaction product prediction model, to enhance the robustness and generalization performance of the reaction product prediction model, and also has strong extendability for a larger data set, for performing a supervision task without relying on manual annotation.

Specifically, as indicated by the task Task4 shown in FIG. 15, reactants R₁₁+R₁₂+R₁₃ and a reaction product P₁₁ in one reaction array R₁₁+R₁₂+R₁₃→P₁₁ are combined with reactants R₂₁+R₂₂+R₂₃ and a reaction product P₂₁ in another reaction array R₂₁+R₂₂+R₂₃→P₂₁, to obtain a composite reaction array, for example, R₁₁+R₁₂+R₁₃+R₂₁+R₂₂+R₂₃→P₁₁+P₂₁.

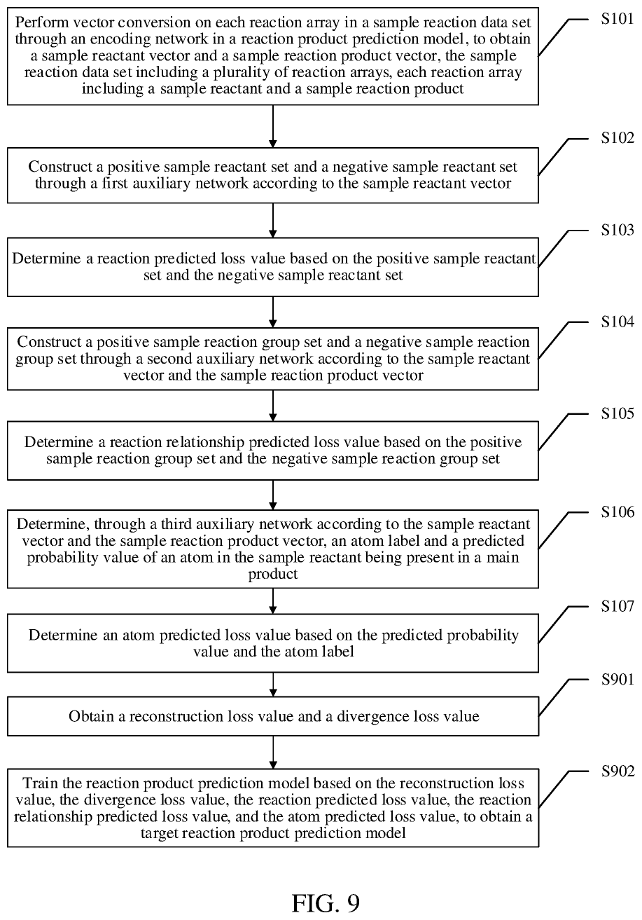

In some embodiments, based on the embodiment corresponding to FIG. 2, in another embodiment of the training method for a reaction product prediction model provided in this application, as shown in FIG. 9, the training the reaction product prediction model based on the reaction predicted loss value, the reaction relationship predicted loss value, and the atom predicted loss value, to obtain a target reaction product prediction model, in step S108 includes the following steps:

Step S901: Obtain a reconstruction loss value and a divergence loss value.

Step S902: Train the reaction product prediction model based on the reconstruction loss value, the divergence loss value, the reaction predicted loss value, the reaction relationship predicted loss value, and the atom predicted loss value, to obtain the target reaction product prediction model.

Specifically, when the VAE architecture satisfying electron transfer conservation shown in FIG. 16 is used as the reaction product prediction model, in this embodiment, a multi-task learning training method can be used, to obtain the reconstruction loss value and the divergence loss value based on the VAE architecture.

Further, objective functions, such as the reconstruction loss value, the divergence loss value, the reaction predicted loss value, the reaction relationship predicted loss value, and the atom predicted loss value, may be directly added to the total objective function in a weighted method based on the following total objective function formula (6), to obtain a total objective function value:

$${L}_{total} = {L}_{reconstruct} + α {L}_{KL} + β {L}_{A} + λ {L}_{B} B + θ {L}_{C} \qquad ( 6 )$$

where Lₜₒₜₐₗ represents the total objective function value, L_{reconstruct} represents the reconstruction loss value, L_{KL} represents the divergence loss value, L_{A} represents the reaction predicted loss value, L_{B} represents the reaction relationship predicted loss value, L_{C} represents the atom predicted loss value, and α, β, Δ, and θ are weight parameters set according to actual application requirements, which are not specifically limited herein.

Further, parameter adjustment may be performed on the reaction product prediction model based on the total objective function value. Specifically, parameter adjustment may be performed using an iterative method of backpropagation or another method, which is not specifically limited herein, to obtain the target reaction product prediction model.

Accordingly, training the reaction product prediction model based on the reconstruction loss value, the divergence loss value, the reaction predicted loss value, the reaction relationship predicted loss value, and the atom predicted loss value helps the trained reaction product prediction model possess better performance, and helps improve the efficiency of training the reaction product prediction model.

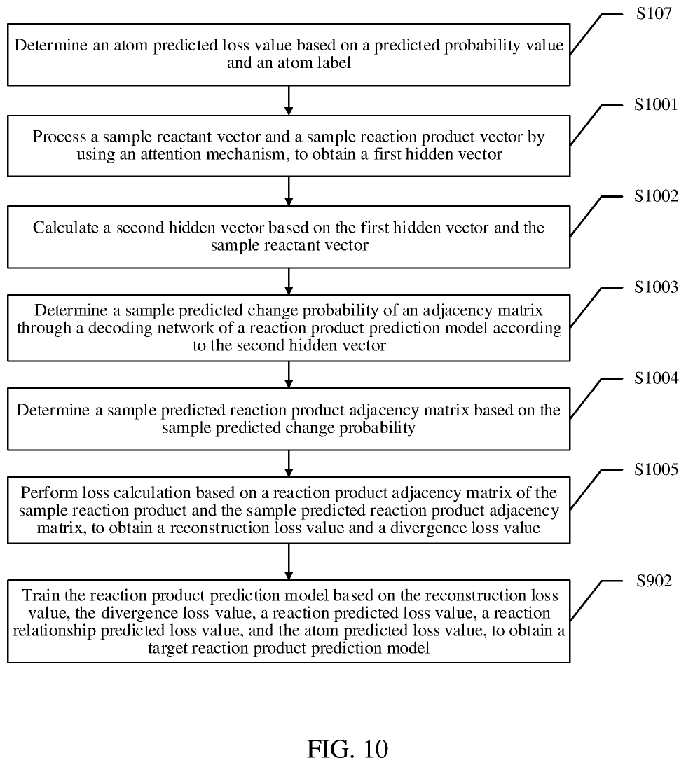

In some embodiments, based on the embodiment corresponding to FIG. 9, in another embodiment of the training method for a reaction product prediction model provided in this application, as shown in FIG. 10, the obtaining a reconstruction loss value and a divergence loss value in step S901 includes the following steps:

Step S1001: Process the sample reactant vector and the sample reaction product vector by using an attention mechanism, to obtain a first hidden vector.

Step S1002: Calculate a second hidden vector modes based on the first hidden vector and the sample reactant vector.

Step S1003: Determine a sample predicted change probability of an adjacency matrix through a decoding network of the reaction product prediction model according to the second hidden vector.

Step S1004: Determine a sample predicted reaction product adjacency matrix based on the sample predicted change probability.

Step S1005: Perform loss calculation based on a reaction product adjacency matrix of the sample reaction product and the sample predicted reaction product adjacency matrix, to obtain the reconstruction loss value and the divergence loss value.

Specifically, as shown in FIG. 16, an attention mechanism is adopted for the sample reactant vector and the sample reaction product vector. That is, a reactant embedding and a product embedding may be sent through a layer of cross attention mechanism. Cross attention may be implemented directly through the transformer decoder based on the following formulas (7), (8), and (9), to obtain parameter vectors μ and log σ of a Gaussian distribution, and in addition, a first hidden vector conforming to the Gaussian distribution is obtained through reparameterization:

$${h}^{z} = Mean ( TransformerDecoder ( {h}^{R} , {h}^{P} ) ) \qquad ( 7 )$$

$$μ = {W}_{μ} Re LU ( {h}^{z} ) + {b}_{μ} \qquad ( 8 )$$

$$log σ = {W}_{σ} Re LU ( {h}^{z} ) + {b}_{σ} \qquad ( 9 )$$

where h^{z} represents the first hidden vector, and W_{μ}, b_{μ}, W_{σ}, and b_{σ} represent model parameters.

Further, a second hidden vector is calculated based on the first hidden vector and the sample reactant vector. Specifically, the first hidden vector and the sample reactant vector are added based on the following formula (10) and caused to pass through a layer of transformer, to obtain a new hidden vector, that is, the second hidden vector h_{L}:

$${h}_{L} = Transformer ( {h}^{R} + {h}^{z} ) . \qquad ( 10 )$$

Further, as shown in FIG. 16, the second hidden vector is input into the decoding network of the reaction product prediction model, and the second hidden vector h_{L} is decoded through the decoding network. The decoding network decoder is mainly formed by two separate self-attention mechanisms. One self-attention mechanism outputs an attention matrix as a probability that electrons shared between two atoms increase, and the other self-attention mechanism outputs an attention matrix as a probability that electrons shared between two atoms decrease. Because transfer of electrons is conserved, this property is modeled using characteristics of the weight matrix of the attention mechanism, where specific decoding operations include the following formulas (11) and (12), to obtain a sample predicted change probability of the adjacency matrix:

$${W}^{+ d} = SelfAttention ( {h}_{L} ) \qquad ( 11 )$$

$${W}^{- d} = SelfAttention ( {h}_{L} ) \qquad ( 12 )$$

where W^{+d} represents a sample predicted change probability, that is, a probability matrix of electrons between each pair of atoms increasing, and W^{-d} represents another sample predicted change probability, that is, a probability matrix of electrons between each pair of atoms decreasing.

Further, a sample predicted reaction product adjacency matrix is calculated based on the sample predicted change probability. Specifically, a sample predicted change amount of an adjacency matrix may be calculated based on the sample predicted change probability, the sample predicted reaction product adjacency matrix may be calculated based on the sample predicted change amount of the adjacency matrix and an adjacency matrix of a sample reactant, and then cross-entropy loss calculation may be performed based on the reaction product adjacency matrix of the sample reaction product and the sample predicted reaction product adjacency matrix, to obtain the reconstruction loss value and the divergence loss value.

Each value in an adjacency matrix in this embodiment is not just 0 or 1 (0 represents that there is no connection between corresponding atom pairs, and 1 represents that there is a connection between corresponding atom pairs), and instead, four values, 0, 1, 2, and 3 respectively represent no connection, a single-bond connection, a double-bond connection, and a triple-bond connection. An aromatic bond connection is represented as 1, and atoms connected by the aromatic bond connection are marked as aromatic atoms, for distinguishing from a single-bond connection.

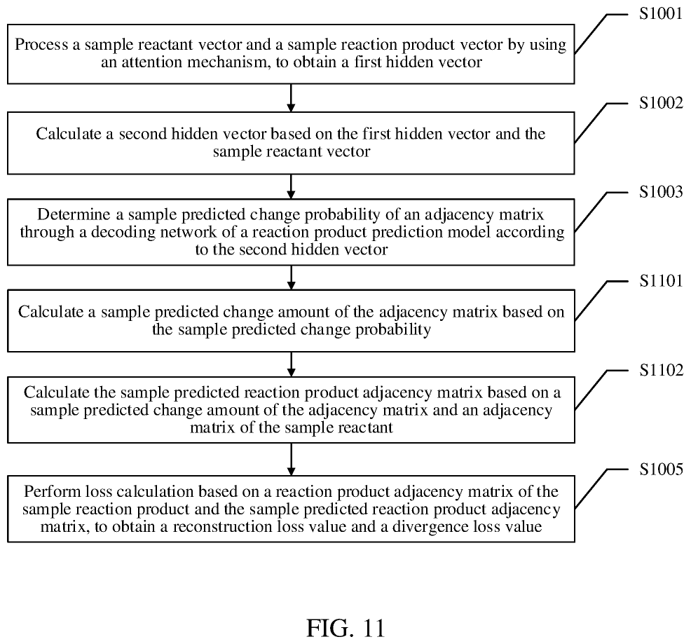

In some embodiments, based on the embodiment corresponding to FIG. 10, in another embodiment of the training method for a reaction product prediction model provided in this application, as shown in FIG. 11, the determining a sample predicted reaction product adjacency matrix based on the sample predicted change probability in step S1004 includes the following steps:

Step S1101: Calculate a sample predicted change amount of the adjacency matrix based on the sample predicted change probability.

Step S1102: Calculate the sample predicted reaction product adjacency matrix based on a sample predicted change amount of the adjacency matrix and an adjacency matrix of the sample reactant.

Specifically, after the sample predicted change probability is obtained, the sample predicted change amount of the adjacency matrix may be calculated based on the sample predicted change probability and using the following formula (13). That is, based on the probability matrix W^{+d} of electrons between each pair of atoms increasing is subtracted from the probability matrix W^{-d} of electrons between each pair of atoms decreasing and multiplied by 4, to obtain the predicted sample predicted change amount ΔA between each pair of atoms:

$$Δ A = ( {W}^{+ d} - {W}^{- d} ) \times 4 \qquad ( 13 )$$

where 4 represents a maximum change amount between each pair of atoms.

Further, the sample predicted reaction product adjacency matrix is calculated based on a sample predicted change amount of the adjacency matrix and an adjacency matrix of the sample reactant and using the following formula (14):

$${A}^{P} = {A}^{R} + Δ A \qquad ( 14 )$$

where A^{P} represents the sample predicted reaction product adjacency matrix, and A^{R} represents the adjacency matrix of the sample reactant.

Further, because the adjacency matrix needs to be symmetric, the sample predicted reaction product adjacency matrix may be symmetrized using the following formula (15):

$${A}^{P} = \frac{{A}^{P} + {( {A}^{P} )}^{T}}{2} . \qquad ( 15 )$$

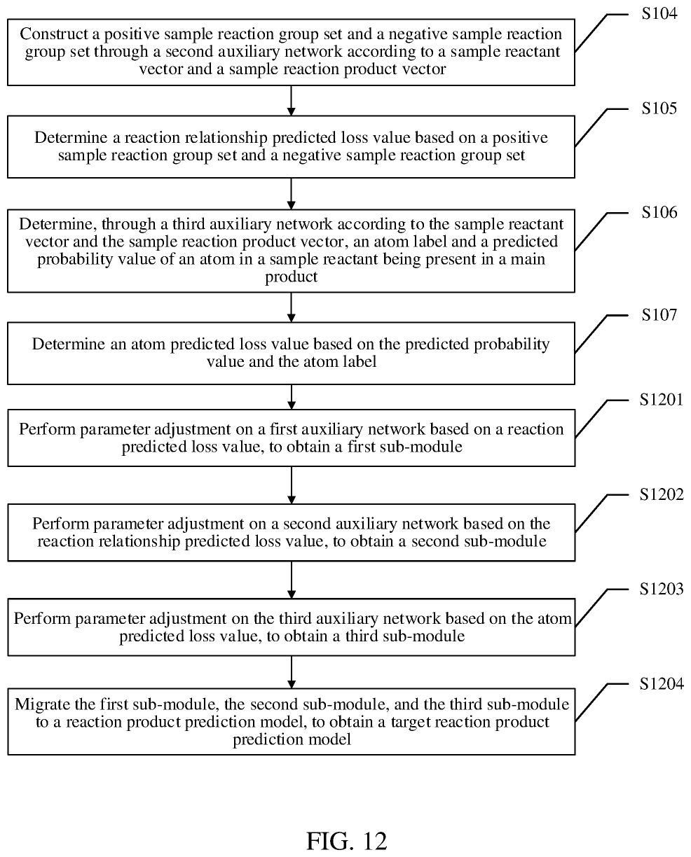

In some embodiments, based on the embodiment corresponding to FIG. 2, in another embodiment of the parameter adjustment method for a reaction product prediction model provided this application, as shown in FIG. 12, the training the reaction product prediction model based on the reaction predicted loss value, the reaction relationship predicted loss value, and the atom predicted loss value, to obtain a target reaction product prediction model, in step S108 includes the following steps:

Step S1201: Perform parameter adjustment on the first auxiliary network based on the reaction predicted loss value, to obtain a first sub-module.

Step S1202: Perform parameter adjustment on the second auxiliary network based on the reaction relationship predicted loss value, to obtain a second sub-module.

Step S1203: Perform parameter adjustment on the third auxiliary network based on the atom predicted loss value, to obtain a third sub-module.

Step S1204: Migrate the first sub-module, the second sub-module, and the third sub-module to the reaction product prediction model, to obtain the target reaction product prediction model.

Specifically, in this embodiment, the first auxiliary network, the second auxiliary network, and the third auxiliary network may be pre-trained using a pre-training policy and based on the reaction predicted loss value, the reaction relationship predicted loss value, and the atom predicted loss value respectively, that is, parameter adjustment is performed on the first auxiliary network based on the reaction predicted loss value to obtain the first sub-module, parameter adjustment is performed on the second auxiliary network based on the reaction relationship predicted loss value to obtain the second sub-module, and parameter adjustment is performed on the third auxiliary network based on the atom predicted loss value to obtain the third sub-module, and then, the first sub-module, the second sub-module, and the third sub-module are migrated to the reaction product prediction model. Specifically, skeleton networks of the first sub-module, the second sub-module and the third sub-module is migrated to the reaction product prediction model, and then, layers outputting embeddings of the skeleton networks are connected to a decoder network layer for reaction product prediction, to obtain the target reaction product prediction model.

Accordingly, the first auxiliary network, the second auxiliary network, and the third auxiliary network are pre-trained respectively based on the pre-training policy, and the trained first auxiliary network, the trained second auxiliary network, and the trained third auxiliary network are migrated to the reaction product prediction model, which can effectively improve the efficiency of training the reaction product prediction model, and also helps improve the performance of the reaction product prediction model.

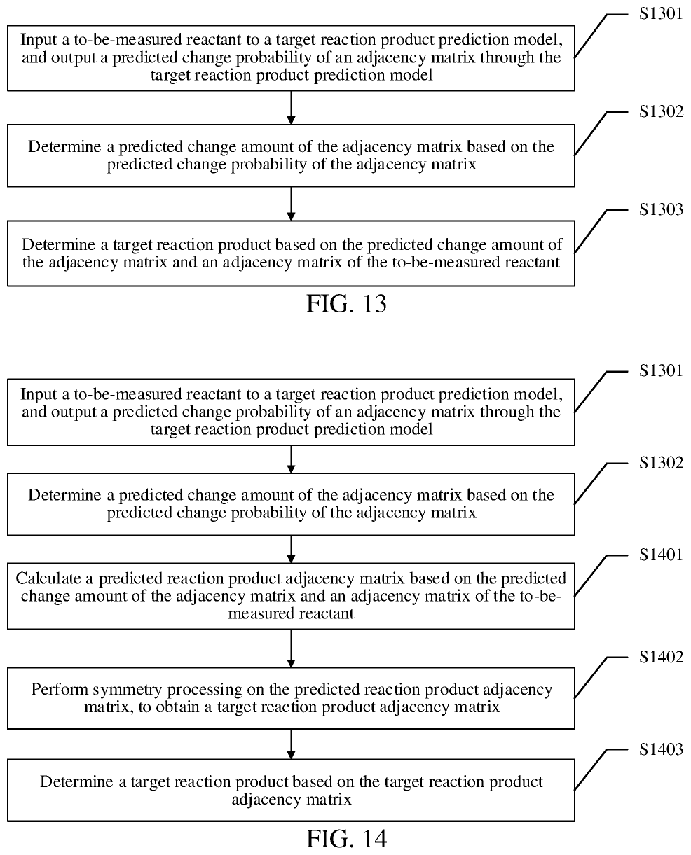

The application method for a reaction product prediction model according to this application is described below. The method may be performed by a computer device. The computer device may be a server or a terminal device. Referring to FIG. 13, an embodiment of the application method for a reaction product prediction model in this application includes the following steps:

Step S1301: Input a to-be-measured reactant to a target reaction product prediction model, and output a predicted change probability of an adjacency matrix through the target reaction product prediction model.

Step S1302: Determine a predicted change amount of the adjacency matrix based on the predicted change probability of the adjacency matrix.

Step S1303: Determine a target reaction product based on the predicted change amount of the adjacency matrix and an adjacency matrix of the to-be-measured reactant.

In this embodiment, after the to-be-measured reactant is obtained, the to-be-measured reactant may be inputted to the target reaction product prediction model, the predicted change probability of the output adjacency matrix may be outputted through the target reaction product prediction model, the predicted change amount of the adjacency matrix may be calculated based on the predicted change probability of the adjacency matrix, and then, the target reaction product may be determined based on the predicted change amount of the adjacency matrix and an adjacency matrix of the to-be-measured reactant, so that the obtained target reaction product can be subsequently applied to scenarios such as scientific pharmaceuticals or drug verification.

It may be understood that the target reaction product prediction model may be used as an effective verification tool for drug retrosynthesis, to improve the efficiency of research on new drug synthesis paths. The target reaction product prediction model may reveal some hidden scientific laws and provide new scientific knowledge. The target reaction product prediction model may provide a prediction more accurate than those provided by experts, and not only can still predict a reliable candidate product when no known template can be used, but also can predict a new reaction, thereby greatly improving the efficiency of researching and development of new drugs and the like.

Specifically, the to-be-measured reactant is inputted to the target reaction product prediction model, and the predicted change probability of the adjacency matrix is outputted through the target reaction product prediction model, that is, based on the probability matrix W^{+d} indicating electrons between each pair of atoms increase and the probability matrix W^{-d} indicating electrons between each pair of atoms decrease, and then, the predicted change amount ΔA of the adjacency matrix may be calculated using the foregoing formula (13).

In some embodiments, based on the embodiment corresponding to FIG. 13, in another embodiment of the application method for a reaction product prediction model provided in this application, as shown in FIG. 14, the determining a target reaction product based on the predicted change amount of the adjacency matrix and an adjacency matrix of the to-be-measured reactant in step S1303 includes the following steps:

Step S1401: Calculate a predicted reaction product adjacency matrix based on the predicted change amount of the adjacency matrix and the adjacency matrix of the to-be-measured reactant.

Step S1402: Perform symmetry processing on the predicted reaction product adjacency matrix, to obtain a target reaction product adjacency matrix.

Step S1403: Determine the target reaction product based on the target reaction product adjacency matrix.

Specifically, the predicted reaction product adjacency matrix may be calculated using the foregoing formula (14) and based on the predicted change amount of the adjacency matrix and an adjacency matrix of the to-be-measured reactant, where A^{P} represents the predicted reaction product adjacency matrix, and A^{R} represents the adjacency matrix of the to-be-measured reactant.

Further, because the adjacency matrix needs to symmetric, symmetry processing may be performed on the predicted reaction product adjacency matrix using the foregoing formula (15), to obtain the target reaction product adjacency matrix, and then, the target reaction product may be calculated based on the target reaction product adjacency matrix A^{P}.

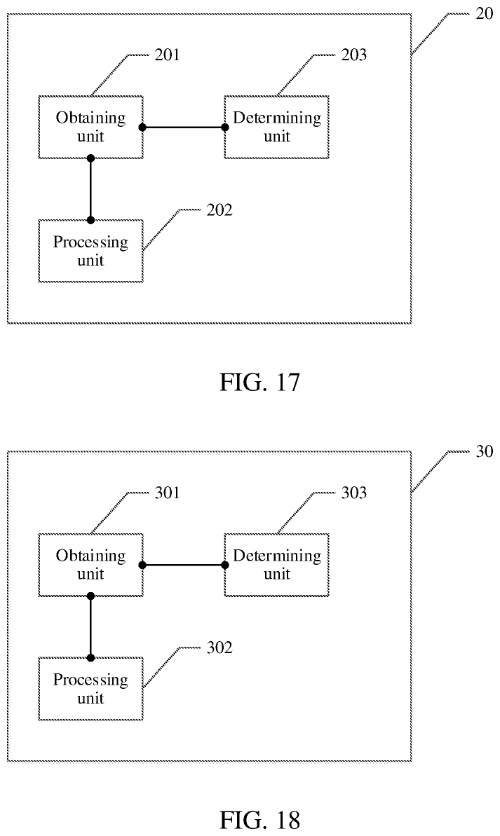

A training apparatus for a reaction product prediction model in this application is described below in detail. FIG. 17 is a schematic diagram of an embodiment of a training apparatus for a reaction product prediction model according to an embodiment of this application. A training apparatus 20 for a reaction product prediction model includes:
an obtaining unit 201, configured to perform vector conversion on each reaction array in a sample reaction data set through an encoding network in a reaction product prediction model, to obtain a sample reactant vector and a sample reaction product vector, the sample reaction data set including a plurality of reaction arrays, each reaction array including a sample reactant and a sample reaction product,
the obtaining unit 201 being further configured to construct a positive sample reactant set and a negative sample reactant set through a first auxiliary network according to the sample reactant vector;
a processing unit 202, configured to determine a reaction predicted loss value based on the positive sample reactant set and the negative sample reactant set,
the obtaining unit 201 being further configured to construct a positive sample reaction group set and a negative sample reaction group set through a second auxiliary network according to the sample reactant vector and the sample reaction product vector;
the processing unit 202 being further configured to determine a reaction relationship predicted loss value based on the positive sample reaction group set and the negative sample reaction group set;
the obtaining unit 201 being further configured to determine, through a third auxiliary network according to the sample reactant vector and the sample reaction product vector, an atom label and a predicted probability value of an atom in the sample reactant being present in a main product; and
the processing unit 202 being further configured to determine an atom predicted loss value based on the predicted probability value and the atom label; and
a determining unit 203, configured to train the reaction product prediction model based on the reaction predicted loss value, the reaction relationship predicted loss value, and the atom predicted loss value, to obtain a target reaction product prediction model.

In some embodiments, based on the foregoing embodiment corresponding to FIG. 17, in another embodiment of the training apparatus for a reaction product prediction model provided in this application:

The processing unit 202 is further configured to perform data augmentation processing on the sample reaction data set, to obtain a sample composite reaction data set.

The processing unit 202 is further configured to summarize the sample reaction data set and the sample composite reaction data set into an extended sample reaction data set.

The obtaining unit 201 may be further configured to perform vector conversion on each reaction array in the extended sample reaction data set through the encoding network in the reaction product prediction model, to obtain the sample reactant vector and the sample reaction product vector.

In some embodiments, based on the foregoing embodiment corresponding to FIG. 17, in another embodiment of the training apparatus for a reaction product prediction model provided in this application, the obtaining unit 201 may be further configured to:
sample the sample reactant vectors corresponding to any two sample reactants from a same reaction array as a positive sample reactant combination, and add the positive sample reactant combination to the positive sample reactant set; and
sample the sample reactant vectors corresponding to any two sample reactants from different reaction arrays as a negative sample reactant combination, and add the negative sample reactant combination to the negative sample reactant set.

In some embodiments, based on the foregoing embodiment corresponding to FIG. 17, in another embodiment of the training apparatus for a reaction product prediction model provided in this application, the obtaining unit 201 may be further configured to:
use the sample reactant vectors and the sample reaction product vectors corresponding to all the reaction arrays as the positive sample reaction group set; and
match the sample reactant vectors and the sample reaction product vectors corresponding to different reaction arrays, to obtain the negative sample reaction group set.

In some embodiments, based on the foregoing embodiment corresponding to FIG. 17, in another embodiment of the training apparatus for a reaction product prediction model provided in this application, the obtaining unit 201 may be further configured to:
predict the sample reactant through the third auxiliary network, to obtain the predicted probability value of the atom in the sample reactant being present in the main product;
compare the atom in the sample reactant and an atom in the sample reaction product based on the sample reactant vector and the sample reaction product vector, to obtain an atom comparison result; and
determine the atom label based on the atom comparison result.

In some embodiments, based on the foregoing embodiment corresponding to FIG. 17, in another embodiment of the training apparatus for a reaction product prediction model provided in this application, the obtaining unit 201 may be further configured to:
perform, for each reaction array, interaction on internal information of each molecule of the sample reactant between atoms, to obtain sample reactant interaction information;
perform interaction between different sample reactants based on the sample reactant interaction information, to obtain the sample reactant vector;
perform, for each reaction array, interaction on internal information of each molecule of the sample reaction product between atoms, to obtain sample reaction product interaction information; and
perform interaction between different sample reaction products based on the sample reaction product interaction information, to obtain the sample reaction product vector.

In some embodiments, based on the foregoing embodiment corresponding to FIG. 17, in another embodiment of the training apparatus for a reaction product prediction model provided in this application, the processing unit 202 may be further configured to:
select two reaction arrays randomly from the sample reaction data set;
combine sample reactants in the two selected reaction arrays, to obtain a sample composite reactant;
combine sample reaction products in the two selected reaction arrays, to obtain a sample composite reaction product; and
obtain a composite reaction array based on the sample composite reactant and the sample composite reaction product, and construct the sample composite reaction data set based on the composite reaction array.

In some embodiments, based on the foregoing embodiment corresponding to FIG. 17, in another embodiment of the training apparatus for a reaction product prediction model provided in this application, the determining unit 203 may be further configured to:
obtain a reconstruction loss value and a divergence loss value; and
train the reaction product prediction model based on the reconstruction loss value, the divergence loss value, the reaction predicted loss value, the reaction relationship predicted loss value, and the atom predicted loss value, to obtain the target reaction product prediction model.

In some embodiments, based on the foregoing embodiment corresponding to FIG. 17, in another embodiment of the training apparatus for a reaction product prediction model provided in this application, the determining unit 203 may be further configured to:
process the sample reactant vector and the sample reaction product vector by using an attention mechanism, to obtain a first hidden vector;
calculate a second hidden vector based on the first hidden vector and the sample reactant vector;
determine a sample predicted change probability of an adjacency matrix through a decoding network of the reaction product prediction model according to the second hidden vector;
determine a sample predicted reaction product adjacency matrix based on the sample predicted change probability; and
perform loss calculation based on a reaction product adjacency matrix of the sample reaction product and the sample predicted reaction product adjacency matrix, to obtain the reconstruction loss value and the divergence loss value.

In some embodiments, based on the foregoing embodiment corresponding to FIG. 17, in another embodiment of the training apparatus for a reaction product prediction model provided in this application, the determining unit 203 may be further configured to:
calculate a sample predicted change amount of the adjacency matrix based on the sample predicted change probability; and
calculate the sample predicted reaction product adjacency matrix based on a sample predicted change amount of the adjacency matrix and an adjacency matrix of the sample reactant.

In some embodiments, based on the foregoing embodiment corresponding to FIG. 17, in another embodiment of the training apparatus for a reaction product prediction model provided in this application, the determining unit 203 may be further configured to:
perform parameter adjustment on the first auxiliary network based on the reaction predicted loss value, to obtain a first sub-module;
perform parameter adjustment on the second auxiliary network based on the reaction relationship predicted loss value, to obtain a second sub-module;
perform parameter adjustment on the third auxiliary network based on the atom predicted loss value, to obtain a third sub-module; and
migrate the first sub-module, the second sub-module, and the third sub-module to the reaction product prediction model, to obtain the target reaction product prediction model.

An application apparatus for a reaction product prediction model in this application is described below in detail. FIG. 18 is a schematic diagram of an embodiment of an application apparatus for a reaction product prediction model according to an embodiment of this application. An application apparatus 30 for a reaction product prediction model includes:
an obtaining unit 301, configured to input a to-be-measured reactant to a target reaction product prediction model, and output a predicted change probability of an adjacency matrix through the target reaction product prediction model, the target reaction product prediction model being obtained through training by the training apparatus for a reaction product prediction model shown in FIG. 17;
a processing unit 302, configured to determine a predicted change amount of the adjacency matrix based on the predicted change probability of the adjacency matrix; and
a determining unit 303, configured to determine a target reaction product based on the predicted change amount of the adjacency matrix and an adjacency matrix of the to-be-measured reactant.

In some embodiments, based on the foregoing embodiment corresponding to FIG. 18, in another embodiment of the application apparatus for a reaction product prediction model provided in this application, the determining unit 303 may be further configured to:
calculate a predicted reaction product adjacency matrix based on the predicted change amount of the adjacency matrix and the adjacency matrix of the to-be-measured reactant;
perform symmetry processing on the predicted reaction product adjacency matrix, to obtain a target reaction product adjacency matrix; and
determine the target reaction product based on the target reaction product adjacency matrix.

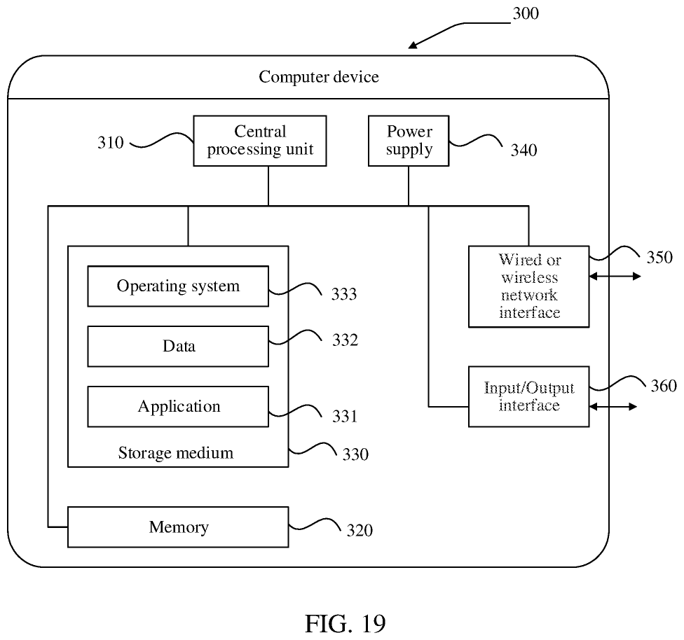

Another aspect of this application provides another schematic diagram of a computer device. FIG. 19 is a schematic structural diagram of a computer device according to an embodiment of this application. The computer device 300 may vary greatly due to different configurations or performance, and may include one or more central processing units (CPU) 310 (for example, one or more processors) and a memory 320, and one or more storage media 330 (for example, one or more mass storage devices) that store applications 331 or data 332. The memory 320 and the storage medium 330 may be transient or persistent storages. The program stored in the storage medium 330 may include one or more modules (not shown in the figure), and each module may include a series of instructions and operations for the computer device 300. Furthermore, the CPUs 310 may be configured to: communicate with the storage media 330, and perform, on the computer device 300, the series of instruction and operations in the storage medium 330.

The computer device 300 may further include one or more power supplies 340, one or more wired or wireless network interfaces 350, one or more input/output interfaces 360, and/or one or more operating systems 333, for example, Windows Server™, Mac OS X™, Unix™ Linux™, or FreeBSD™.

The foregoing computer device 300 is further configured to perform the steps in the embodiments corresponding to FIG. 2 to FIG. 13 and the steps in the embodiment corresponding to FIG. 14.

Another aspect of this application provides a computer-readable storage medium, having a computer program stored therein. When the computer program is executed by a processor, the steps in the method described in the embodiments shown in FIG. 2 to FIG. 13 and the steps in the method described in the embodiment shown in FIG. 14 are implemented.

Another aspect of this application provides a computer program product including a computer program. When the computer program is executed by a processor, the steps in the method described in the embodiments shown in FIG. 2 to FIG. 13 and the steps in the method described in the embodiment shown in FIG. 14 are implemented.

A person skilled in the art may clearly understand that, for the purpose of convenient and brief description, for a detailed working process of the system, apparatus, and unit described above, refer to a corresponding process in the method embodiments, and details are not described herein again.

In the several embodiments provided in this application, it is to be understood that the disclosed system, apparatus, and method may be implemented in other methods. For example, the described apparatus embodiment is merely an example. For example, the unit division is merely logical function division and may be another division in some embodiments. For example, a plurality of units or components may be combined or integrated into another system, or some features may be ignored or not performed. In addition, the displayed or discussed mutual couplings or direct couplings or communication connections may be implemented by using some interfaces. The indirect couplings or communication connections between the apparatuses or units may be implemented in electric, mechanical, or other forms.

The units described as separate parts may or may not be physically separate, and parts displayed as units may or may not be physical units, may be located in one position, or may be distributed on a plurality of network units. Some or all of the units may be selected based on actual requirements to achieve the objectives of the solutions of embodiments.

In addition, functional units in embodiments of this application may be integrated into one processing unit, or each of the units may exist alone physically, or two or more units are integrated into one unit. The integrated unit may be implemented in a form of hardware, or may be implemented in a form of a software functional unit.

When the integrated unit is implemented in the form of a software functional unit and sold or used as an independent product, the integrated unit may be stored in a computer-readable storage medium. Based on such an understanding, the technical solutions of this application essentially, or the part contributing to the conventional technology, or all or some of the technical solutions may be implemented in the form of a software product. The computer software product is stored in a storage medium, and includes several instructions for enabling a computer device (which may be a personal computer, a server, or a network device) to perform all or some of the steps of the methods described in embodiments of this application. The foregoing storage medium includes any medium that can store program code, for example, a USB flash drive, a removable hard disk, a read-only memory (ROM), a random access memory (RAM), a magnetic disk, or an optical disc.

What is claimed is:

1. A training method for a reaction product prediction model, performed by a computer device, the method comprising:
performing vector conversion on each reaction array in a sample reaction data set through an encoding network in a reaction product prediction model, to obtain a sample reactant vector and a sample reaction product vector, the sample reaction data set comprising a plurality of reaction arrays, each reaction array comprising a sample reactant and a sample reaction product;
constructing a positive sample reactant set and a negative sample reactant set through a first auxiliary network according to the sample reactant vector;
determining a reaction predicted loss value based on the positive sample reactant set and the negative sample reactant set;
constructing a positive sample reaction group set and a negative sample reaction group set through a second auxiliary network according to the sample reactant vector and the sample reaction product vector;
determining a reaction relationship predicted loss value based on the positive sample reaction group set and the negative sample reaction group set;
determining, through a third auxiliary network according to the sample reactant vector and the sample reaction product vector, an atom label and a predicted probability value of an atom in the sample reactant being present in a main product;
determining an atom predicted loss value based on the predicted probability value and the atom label; and
training the reaction product prediction model based on the reaction predicted loss value, the reaction relationship predicted loss value, and the atom predicted loss value, to obtain a target reaction product prediction model.

2. The method according to claim 1, wherein before the performing vector conversion on each reaction array in a sample reaction data set through an encoding network in a reaction product prediction model, to obtain a sample reactant vector and a sample reaction product vector, the method further comprises:
performing data augmentation processing on the sample reaction data set, to obtain a sample composite reaction data set; and
summarizing the sample reaction data set and the sample composite reaction data set into an extended sample reaction data set; and
the performing vector conversion on each reaction array in a sample reaction data set through an encoding network in a reaction product prediction model, to obtain a sample reactant vector and a sample reaction product vector, comprises:
performing vector conversion on each reaction array in the extended sample reaction data set through the encoding network in the reaction product prediction model, to obtain the sample reactant vector and the sample reaction product vector.

3. The method according to claim 1, wherein the constructing a positive sample reactant set and a negative sample reactant set through a first auxiliary network according to the sample reactant vector comprises:
sampling the sample reactant vectors corresponding to any two sample reactants from a same reaction array as a positive sample reactant combination, and adding the positive sample reactant combination to the positive sample reactant set; and
sampling the sample reactant vectors corresponding to any two sample reactants from different reaction arrays as a negative sample reactant combination, and adding the negative sample reactant combination to the negative sample reactant set.

4. The method according to claim 1, wherein the constructing a positive sample reaction group set and a negative sample reaction group set through a second auxiliary network according to the sample reactant vector and the sample reaction product vector comprises:
using the sample reactant vectors and the sample reaction product vectors corresponding to all the reaction arrays as the positive sample reaction group set; and
matching the sample reactant vectors and the sample reaction product vectors corresponding to different reaction arrays, to obtain the negative sample reaction group set.

5. The method according to claim 1, wherein the determining, through a third auxiliary network according to the sample reactant vector and the sample reaction product vector, an atom label and a predicted probability value of an atom in the sample reactant being present in a main product comprises:
predicting the sample reactant through the third auxiliary network, to obtain the predicted probability value of the atom in the sample reactant being present in the main product;
comparing the atom in the sample reactant and an atom in the sample reaction product based on the sample reactant vector and the sample reaction product vector, to obtain an atom comparison result; and
determining the atom label based on the atom comparison result.

6. The method according to claim 1, wherein the performing vector conversion on each reaction array in a sample reaction data set through an encoding network in a reaction product prediction model, to obtain a sample reactant vector and a sample reaction product vector, comprises:
performing, for each reaction array, interaction on internal information of each molecule of the sample reactant between atoms, to obtain sample reactant interaction information;
performing interaction between different sample reactants based on the sample reactant interaction information, to obtain the sample reactant vector;
performing, for each reaction array, interaction on internal information of each molecule of the sample reaction product between atoms, to obtain sample reaction product interaction information; and
performing interaction between different sample reaction products based on the sample reaction product interaction information, to obtain the sample reaction product vector.

7. The method according to claim 2, wherein the performing data augmentation processing on the sample reaction data set, to obtain a sample composite reaction data set, comprises:
selecting two reaction arrays randomly from the sample reaction data set;
combining sample reactants in the two selected reaction arrays, to obtain a sample composite reactant;
combining sample reaction products in the two selected reaction arrays, to obtain a sample composite reaction product; and
obtaining a composite reaction array based on the sample composite reactant and the sample composite reaction product, and constructing the sample composite reaction data set based on the composite reaction array.

8. The method according to claim 1, wherein the training the reaction product prediction model based on the reaction predicted loss value, the reaction relationship predicted loss value, and the atom predicted loss value, to obtain a target reaction product prediction model, comprises:
obtaining a reconstruction loss value and a divergence loss value; and
training the reaction product prediction model based on the reconstruction loss value, the divergence loss value, the reaction predicted loss value, the reaction relationship predicted loss value, and the atom predicted loss value, to obtain the target reaction product prediction model.

9. The method according to claim 8, wherein the obtaining a reconstruction loss value and a divergence loss value comprises:
processing the sample reactant vector and the sample reaction product vector by using an attention mechanism, to obtain a first hidden vector;
calculating a second hidden vector based on the first hidden vector and the sample reactant vector;
determining a sample predicted change probability of an adjacency matrix through a decoding network of the reaction product prediction model according to the second hidden vector;
determining a sample predicted reaction product adjacency matrix based on the sample predicted change probability; and
performing loss calculation based on a reaction product adjacency matrix of the sample reaction product and the sample predicted reaction product adjacency matrix, to obtain the reconstruction loss value and the divergence loss value.

10. The method according to claim 9, wherein the determining a sample predicted reaction product adjacency matrix based on the sample predicted change probability comprises:
calculating a sample predicted change amount of the adjacency matrix based on the sample predicted change probability; and
calculating the sample predicted reaction product adjacency matrix based on a sample predicted change amount of the adjacency matrix and an adjacency matrix of the sample reactant.

11. The method according to claim 1, wherein the training the reaction product prediction model based on the reaction predicted loss value, the reaction relationship predicted loss value, and the atom predicted loss value, to obtain a target reaction product prediction model, comprises:
performing parameter adjustment on the first auxiliary network based on the reaction predicted loss value, to obtain a first sub-module;
performing parameter adjustment on the second auxiliary network based on the reaction relationship predicted loss value, to obtain a second sub-module;
performing parameter adjustment on the third auxiliary network based on the atom predicted loss value, to obtain a third sub-module; and
migrating the first sub-module, the second sub-module, and the third sub-module to the reaction product prediction model, to obtain the target reaction product prediction model.

12. An application method for a reaction product prediction model, performed by a computer device, the method comprising:
inputting a to-be-measured reactant to the target reaction product prediction model, and outputting a predicted change probability of an adjacency matrix through the target reaction product prediction model;
determining a predicted change amount of the adjacency matrix based on the predicted change probability of the adjacency matrix; and
determining a target reaction product based on the predicted change amount of the adjacency matrix and an adjacency matrix of the to-be-measured reactant, wherein the target reaction product prediction model is trained by:
performing vector conversion on each reaction array in a sample reaction data set through an encoding network in a reaction product prediction model, to obtain a sample reactant vector and a sample reaction product vector, the sample reaction data set comprising a plurality of reaction arrays, each reaction array comprising a sample reactant and a sample reaction product;
constructing a positive sample reactant set and a negative sample reactant set through a first auxiliary network according to the sample reactant vector;
determining a reaction predicted loss value based on the positive sample reactant set and the negative sample reactant set;
constructing a positive sample reaction group set and a negative sample reaction group set through a second auxiliary network according to the sample reactant vector and the sample reaction product vector;
determining a reaction relationship predicted loss value based on the positive sample reaction group set and the negative sample reaction group set;
determining, through a third auxiliary network according to the sample reactant vector and the sample reaction product vector, an atom label and a predicted probability value of an atom in the sample reactant being present in a main product;
determining an atom predicted loss value based on the predicted probability value and the atom label; and
training the target reaction product prediction model based on the reaction predicted loss value, the reaction relationship predicted loss value, and the atom predicted loss value, to obtain a target reaction product prediction model.

13. The method according to claim 12, wherein the determining a target reaction product based on the predicted change amount of the adjacency matrix and an adjacency matrix of the to-be-measured reactant comprises:
calculating a predicted reaction product adjacency matrix based on the predicted change amount of the adjacency matrix and the adjacency matrix of the to-be-measured reactant;
performing symmetry processing on the predicted reaction product adjacency matrix, to obtain a target reaction product adjacency matrix; and
determining the target reaction product based on the target reaction product adjacency matrix.

14. A non-transitory computer-readable storage medium, having a computer program stored therein, the computer program, when executed by a processor, implementing a method for training a reaction product prediction model comprising:
performing vector conversion on each reaction array in a sample reaction data set through an encoding network in a reaction product prediction model, to obtain a sample reactant vector and a sample reaction product vector, the sample reaction data set comprising a plurality of reaction arrays, each reaction array comprising a sample reactant and a sample reaction product;
constructing a positive sample reactant set and a negative sample reactant set through a first auxiliary network according to the sample reactant vector;
determining a reaction predicted loss value based on the positive sample reactant set and the negative sample reactant set;
constructing a positive sample reaction group set and a negative sample reaction group set through a second auxiliary network according to the sample reactant vector and the sample reaction product vector;
determining a reaction relationship predicted loss value based on the positive sample reaction group set and the negative sample reaction group set;
determining, through a third auxiliary network according to the sample reactant vector and the sample reaction product vector, an atom label and a predicted probability value of an atom in the sample reactant being present in a main product;
determining an atom predicted loss value based on the predicted probability value and the atom label; and
training the reaction product prediction model based on the reaction predicted loss value, the reaction relationship predicted loss value, and the atom predicted loss value, to obtain a target reaction product prediction model.

15. The computer-readable storage medium according to claim 14, wherein before the performing vector conversion on each reaction array in a sample reaction data set through an encoding network in a reaction product prediction model, to obtain a sample reactant vector and a sample reaction product vector, the method further comprises:
performing data augmentation processing on the sample reaction data set, to obtain a sample composite reaction data set; and
summarizing the sample reaction data set and the sample composite reaction data set into an extended sample reaction data set; and
the performing vector conversion on each reaction array in a sample reaction data set through an encoding network in a reaction product prediction model, to obtain a sample reactant vector and a sample reaction product vector, comprises:
performing vector conversion on each reaction array in the extended sample reaction data set through the encoding network in the reaction product prediction model, to obtain the sample reactant vector and the sample reaction product vector.

16. The computer-readable storage medium according to claim 14, wherein the constructing a positive sample reactant set and a negative sample reactant set through a first auxiliary network according to the sample reactant vector comprises:
sampling the sample reactant vectors corresponding to any two sample reactants from a same reaction array as a positive sample reactant combination, and adding the positive sample reactant combination to the positive sample reactant set; and
sampling the sample reactant vectors corresponding to any two sample reactants from different reaction arrays as a negative sample reactant combination, and adding the negative sample reactant combination to the negative sample reactant set.

17. The computer-readable storage medium according to claim 14, wherein the constructing a positive sample reaction group set and a negative sample reaction group set through a second auxiliary network according to the sample reactant vector and the sample reaction product vector comprises:
using the sample reactant vectors and the sample reaction product vectors corresponding to all the reaction arrays as the positive sample reaction group set; and
matching the sample reactant vectors and the sample reaction product vectors corresponding to different reaction arrays, to obtain the negative sample reaction group set.

18. The computer-readable storage medium according to claim 14, wherein the determining, through a third auxiliary network according to the sample reactant vector and the sample reaction product vector, an atom label and a predicted probability value of an atom in the sample reactant being present in a main product comprises:
predicting the sample reactant through the third auxiliary network, to obtain the predicted probability value of the atom in the sample reactant being present in the main product;
comparing the atom in the sample reactant and an atom in the sample reaction product based on the sample reactant vector and the sample reaction product vector, to obtain an atom comparison result; and
determining the atom label based on the atom comparison result.

19. The computer-readable storage medium according to claim 14, wherein the performing vector conversion on each reaction array in a sample reaction data set through an encoding network in a reaction product prediction model, to obtain a sample reactant vector and a sample reaction product vector, comprises:
performing, for each reaction array, interaction on internal information of each molecule of the sample reactant between atoms, to obtain sample reactant interaction information;
performing interaction between different sample reactants based on the sample reactant interaction information, to obtain the sample reactant vector;
performing, for each reaction array, interaction on internal information of each molecule of the sample reaction product between atoms, to obtain sample reaction product interaction information; and
performing interaction between different sample reaction products based on the sample reaction product interaction information, to obtain the sample reaction product vector.

20. The computer-readable storage medium according to claim 15, wherein the performing data augmentation processing on the sample reaction data set, to obtain a sample composite reaction data set, comprises:
selecting two reaction arrays randomly from the sample reaction data set;
combining sample reactants in the two selected reaction arrays, to obtain a sample composite reactant;
combining sample reaction products in the two selected reaction arrays, to obtain a sample composite reaction product; and
obtaining a composite reaction array based on the sample composite reactant and the sample composite reaction product, and constructing the sample composite reaction data set based on the composite reaction array.
